SEQUENCE LISTING (156 sequences)

SEQ ID NO: 1 - protein, 9 aa, SARS-CoV-2 Virus
KYFKNHTSP

SEQ ID NO: 2 - protein, 4 aa, SARS-CoV-2 Virus
TTKR

SEQ ID NO: 3 - protein, 10 aa, SARS-CoV-2 Virus
YYHKNNKSWM

SEQ ID NO: 4 - protein, 5 aa, SARS-CoV-2 Virus
ASTEK

SEQ ID NO: 5 - protein, 6 aa, SARS-CoV-2 Virus
AWNRKR

SEQ ID NO: 6 - protein, 7 aa, SARS-CoV-2 Virus
EQDKNTQ

SEQ ID NO: 7 - protein, 6 aa, SARS-CoV-2 Virus
GTNTSN

SEQ ID NO: 8 - protein, 7 aa, SARS-CoV-2 Virus
KYNENGT

SEQ ID NO: 9 - protein, 6 aa, SARS-CoV-2 Virus
LDSKTQ

SEQ ID NO: 10 - protein, 4 aa, SARS-CoV-2 Virus
PKKS

SEQ ID NO: 11 - protein, 12 aa, SARS-CoV-2 Virus
YQTQTNSPRRAR

SEQ ID NO: 12 - protein, 4 aa, SARS-CoV-2 Virus
TKRT

SEQ ID NO: 13 - protein, 6 aa, SARS-CoV-2 Virus
DEDDSE

SEQ ID NO: 14 - protein, 10 aa, SARS-CoV-2 Virus
GYQPYRVVVL

SEQ ID NO: 15 - protein, 10 aa, SARS-CoV-2 Virus
QPYRVVVLSF

SEQ ID NO: 16 - protein, 9 aa, SARS-CoV-2 Virus
PYRVVVLSF

SEQ ID NO: 17 - protein, 8 aa, SARS-CoV-2 Virus
ILLNKHID

SEQ ID NO: 18 - protein, 18 aa, SARS-CoV-2 Virus
AFFGMSRIGMEVTPSGTW

SEQ ID NO: 19 - protein, 10 aa, SARS-CoV-2 Virus
MEVTPSGTWL

SEQ ID NO: 20 - protein, 9 aa, SARS-CoV-2 Virus
GMSRIGMEV

SEQ ID NO: 21 - protein, 9 aa, SARS-CoV-2 Virus
ILLNKHIDA

SEQ ID NO: 22 - protein, 9 aa, SARS-CoV-2 Virus
ALNTPKDHI

SEQ ID NO: 23 - protein, 17 aa, SARS-CoV-2 Virus
IRQGTDYKHWPQIAQFA

SEQ ID NO: 24 - protein, 17 aa, SARS-CoV-2 Virus
KHWPQIAQFAPSASAFF

SEQ ID NO: 25 - protein, 9 aa, SARS-CoV-2 Virus
LALLLLDRL

SEQ ID NO: 26 - protein, 9 aa, SARS-CoV-2 Virus
LLLDRLNQL

SEQ ID NO: 27 - protein, 18 aa, SARS-CoV-2 Virus
LLNKHIDAYKTFPPTEPK

SEQ ID NO: 28 - protein, 9 aa, SARS-CoV-2 Virus
LQLPQGTTL

SEQ ID NO: 29 - protein, 15 aa, SARS-CoV-2 Virus
AQFAPSASAFFGMSR

SEQ ID NO: 30 - protein, 18 aa, SARS-CoV-2 Virus
AQFAPSASAFFGMSRIGM

SEQ ID NO: 31 - protein, 15 aa, SARS-CoV-2 Virus
RRPQGLPNNTASWFT

SEQ ID NO: 32 - protein, 16 aa, SARS-CoV-2 Virus
YKTFPPTEPKKDKKKK

SEQ ID NO: 33 - protein, 16 aa, SARS-CoV-2 Virus
GAALQIPFAMQMAYRF

SEQ ID NO: 34 - protein, 16 aa, SARS-CoV-2 Virus
MAYRFNGIGVTQNVLY

SEQ ID NO: 35 - protein, 18 aa, SARS-CoV-2 Virus
QLIRAAEIRASANLAATK

SEQ ID NO: 36 - protein, 9 aa, SARS-CoV-2 Virus
FIAGLIAIV

SEQ ID NO: 37 - protein, 9 aa, SARS-CoV-2 Virus
ALNTLVKQL

SEQ ID NO: 38 - protein, 9 aa, SARS-CoV-2 Virus
LITGRLQSL

SEQ ID NO: 39 - protein, 9 aa, SARS-CoV-2 Virus
NLNESLIDL

SEQ ID NO: 40 - protein, 17 aa, SARS-CoV-2 Virus
QALNTLVKQLSSNFGAI

SEQ ID NO: 41 - protein, 9 aa, SARS-CoV-2 Virus
RLNEVAKNL

SEQ ID NO: 42 - protein, 9 aa, SARS-CoV-2 Virus
VLNDILSRL

SEQ ID NO: 43 - protein, 9 aa, SARS-CoV-2 Virus
VVFLHVTYV

SEQ ID NO: 44 - protein, 9 aa, SARS-CoV-2 Virus
FIAGLIAIV

SEQ ID NO: 45 - protein, 10 aa, SARS-CoV-2 Virus
GLIAIVMVTI

SEQ ID NO: 46 - protein, 9 aa, SARS-CoV-2 Virus
IITTDNTFV

SEQ ID NO: 47 - protein, 9 aa, SARS-CoV-2 Virus
ALNTLVKQL

SEQ ID NO: 48 - protein, 9 aa, SARS-CoV-2 Virus
LITGRLQSL

SEQ ID NO: 49 - protein, 9 aa, SARS-CoV-2 Virus
LLLQYGSFC

SEQ ID NO: 50 - protein, 8 aa, SARS-CoV-2 Virus
LQYGSFCT

SEQ ID NO: 51 - protein, 9 aa, SARS-CoV-2 Virus
NLNESLIDL

SEQ ID NO: 52 - protein, 9 aa, SARS-CoV-2 Virus
RLDKVEAEV

SEQ ID NO: 53 - protein, 9 aa, SARS-CoV-2 Virus
RLNEVAKNL

SEQ ID NO: 54 - protein, 9 aa, SARS-CoV-2 Virus
RLQSLQTYV

SEQ ID NO: 55 - protein, 9 aa, SARS-CoV-2 Virus
VLNDILSRL

SEQ ID NO: 56 - protein, 9 aa, SARS-CoV-2 Virus
VVFLHVTYV

SEQ ID NO: 57 - protein, 8 aa, SARS-CoV-2 Virus
ILLNKHID

SEQ ID NO: 58 - protein, 9 aa, SARS-CoV-2 Virus
FPRGQGVPI

SEQ ID NO: 59 - protein, 9 aa, SARS-CoV-2 Virus
LLLLDRLNQ

SEQ ID NO: 60 - protein, 9 aa, SARS-CoV-2 Virus
GMSRIGMEV

SEQ ID NO: 61 - protein, 9 aa, SARS-CoV-2 Virus
ILLNKHIDA

SEQ ID NO: 62 - protein, 9 aa, SARS-CoV-2 Virus
ALNTPKDHI

SEQ ID NO: 63 - protein, 9 aa, SARS-CoV-2 Virus
LALLLLDRL

SEQ ID NO: 64 - protein, 9 aa, SARS-CoV-2 Virus
LLLDRLNQL

SEQ ID NO: 65 - protein, 10 aa, SARS-CoV-2 Virus
LLLLDRLNQL

SEQ ID NO: 66 - protein, 9 aa, SARS-CoV-2 Virus
LQLPQGTTL

SEQ ID NO: 67 - protein, 9 aa, SARS-CoV-2 Virus
AQFAPSASA

SEQ ID NO: 68 - protein, 9 aa, SARS-CoV-2 Virus
TTLPKGFYA

SEQ ID NO: 69 - protein, 10 aa, SARS-CoV-2 Virus
VLQLPQGTTL

SEQ ID NO: 70 - protein, 10 aa, SARS-CoV-2 Virus
GYQPYRVVVL

SEQ ID NO: 71 - protein, 9 aa, SARS-CoV-2 Virus
PYRVVVLSF

SEQ ID NO: 72 - protein, 9 aa, SARS-CoV-2 Virus
LSPRWYFYY

SEQ ID NO: 73 - protein, 10 aa, SARS-CoV-2 Virus
DSFKEELDKY

SEQ ID NO: 74 - protein, 10 aa, SARS-CoV-2 Virus
LIDLQELGKY

SEQ ID NO: 75 - protein, 9 aa, SARS-CoV-2 Virus
PYRVVVLSF

SEQ ID NO: 76 - protein, 9 aa, SARS-CoV-2 Virus
GTTLPKGFY

SEQ ID NO: 77 - protein, 10 aa, SARS-CoV-2 Virus
VTPSGTWLTY

SEQ ID NO: 78 - protein, 9 aa, SARS-CoV-2 Virus
GSFCTQLNR

SEQ ID NO: 79 - protein, 9 aa, SARS-CoV-2 Virus
GVVFLHVTY

SEQ ID NO: 80 - protein, 9 aa, SARS-CoV-2 Virus
AQALNTLVK

SEQ ID NO: 81 - protein, 9 aa, SARS-CoV-2 Virus
MTSCCSCLK

SEQ ID NO: 82 - protein, 9 aa, SARS-CoV-2 Virus
ASANLAATK

SEQ ID NO: 83 - protein, 10 aa, SARS-CoV-2 Virus
SLIDLQELGK

SEQ ID NO: 84 - protein, 9 aa, SARS-CoV-2 Virus
SVLNDILSR

SEQ ID NO: 85 - protein, 10 aa, SARS-CoV-2 Virus
TQNVLYENQK

SEQ ID NO: 86 - protein, 10 aa, SARS-CoV-2 Virus
CMTSCCSCLK

SEQ ID NO: 87 - protein, 10 aa, SARS-CoV-2 Virus
VQIDRLITGR

SEQ ID NO: 88 - protein, 9 aa, SARS-CoV-2 Virus
KTFPPTEPK

SEQ ID NO: 89 - protein, 10 aa, SARS-CoV-2 Virus
KTFPPTEPKK

SEQ ID NO: 90 - protein, 9 aa, SARS-CoV-2 Virus
LSPRWYFYY

SEQ ID NO: 91 - protein, 9 aa, SARS-CoV-2 Virus
ASAFFGMSR

SEQ ID NO: 92 - protein, 10 aa, SARS-CoV-2 Virus
ATEGALNTPK

SEQ ID NO: 93 - protein, 10 aa, SARS-CoV-2 Virus
QLPQGTTLPK

SEQ ID NO: 94 - protein, 9 aa, SARS-CoV-2 Virus
QQQGQTVTK

SEQ ID NO: 95 - protein, 10 aa, SARS-CoV-2 Virus
QQQQGQTVTK

SEQ ID NO: 96 - protein, 10 aa, SARS-CoV-2 Virus
SASAFFGMSR

SEQ ID NO: 97 - protein, 10 aa, SARS-CoV-2 Virus
SQASSRSSSR

SEQ ID NO: 98 - protein, 9 aa, SARS-CoV-2 Virus
TPSGTWLTY

SEQ ID NO: 99 - protein, 10 aa, SARS-CoV-2 Virus
FPNITNLCPF

SEQ ID NO: 100 - protein, 10 aa, SARS-CoV-2 Virus
APHGVVFLHV

SEQ ID NO: 101 - protein, 9 aa, SARS-CoV-2 Virus
FPRGQGVPI

SEQ ID NO: 102 - protein, 10 aa, SARS-CoV-2 Virus
APSASAFFGM

SEQ ID NO: 103 - protein, 15 aa, SARS-CoV-2 Virus
GAALQIPFAMQMAYR

SEQ ID NO: 104 - protein, 15 aa, SARS-CoV-2 Virus
GWTFGAGAALQIPFA

SEQ ID NO: 105 - protein, 15 aa, SARS-CoV-2 Virus
IDRLITGRLQSLQTY

SEQ ID NO: 106 - protein, 15 aa, SARS-CoV-2 Virus
ISGINASVVNIQKEI

SEQ ID NO: 107 - protein, 15 aa, SARS-CoV-2 Virus
LDKYFKNHTSPDVDL

SEQ ID NO: 108 - protein, 15 aa, SARS-CoV-2 Virus
LGDISGINASVVNIQ

SEQ ID NO: 109 - protein, 15 aa, SARS-CoV-2 Virus
LGFIAGLIAIVMVTI

SEQ ID NO: 110 - protein, 15 aa, SARS-CoV-2 Virus
LNTLVKQLSSNFGAI

SEQ ID NO: 111 - protein, 15 aa, SARS-CoV-2 Virus
LQDVVNQNAQALNTL

SEQ ID NO: 112 - protein, 15 aa, SARS-CoV-2 Virus
LQSLQTYVTQQLIRA

SEQ ID NO: 113 - protein, 15 aa, SARS-CoV-2 Virus
LQTYVTQQLIRAAEI

SEQ ID NO: 114 - protein, 15 aa, SARS-CoV-2 Virus
AQKFNGLTVLPPLLT

SEQ ID NO: 115 - protein, 15 aa, SARS-CoV-2 Virus
PCSFGGVSVITPGTN

SEQ ID NO: 116 - protein, 15 aa, SARS-CoV-2 Virus
QIPFAMQMAYRFNGI

SEQ ID NO: 117 - protein, 15 aa, SARS-CoV-2 Virus
QQLIRAAEIRASANL

SEQ ID NO: 118 - protein, 15 aa, SARS-CoV-2 Virus
QTYVTQQLIRAAEIR

SEQ ID NO: 119 - protein, 15 aa, SARS-CoV-2 Virus
AYRFNGIGVTQNVLY

SEQ ID NO: 120 - protein, 15 aa, SARS-CoV-2 Virus
SSNFGAISSVLNDIL

SEQ ID NO: 121 - protein, 15 aa, SARS-CoV-2 Virus
TGRLQSLQTYVTQQL

SEQ ID NO: 122 - protein, 15 aa, SARS-CoV-2 Virus
WLGFIAGLIAIVMVT

SEQ ID NO: 123 - protein, 15 aa, SARS-CoV-2 Virus
CVNFNFNGLTGTGVL

SEQ ID NO: 124 - protein, 15 aa, SARS-CoV-2 Virus
DKYFKNHTSPDVDLG

SEQ ID NO: 125 - protein, 15 aa, SARS-CoV-2 Virus
IDAYKTFPPTEPKKD

SEQ ID NO: 126 - protein, 15 aa, SARS-CoV-2 Virus
MSRIGMEVTPSGTWL

SEQ ID NO: 127 - protein, 15 aa, SARS-CoV-2 Virus
NKHIDAYKTFPPTEP

SEQ ID NO: 128 - protein, 15 aa, SARS-CoV-2 Virus
VLQLPQGTTLPKGFY

SEQ ID NO: 129 - protein, 9 aa, SARS-CoV-2 Virus
LQIPFAMQM

SEQ ID NO: 130 - protein, 9 aa, SARS-CoV-2 Virus
RVDFCGKGY

SEQ ID NO: 131 - protein, 10 aa, SARS-CoV-2 Virus
YEQYIKWPWY

SEQ ID NO: 132 - protein, 9 aa, SARS-CoV-2 Virus
GRLQSLQTY

SEQ ID NO: 133 - protein, 9 aa, SARS-CoV-2 Virus
VRFPNITNL

SEQ ID NO: 134 - protein, 17 aa, SARS-CoV-2 Virus
DVVNQNAQALNTLVKQL

SEQ ID NO: 135 - protein, 17 aa, SARS-CoV-2 Virus
EAEVQIDRLITGRLQSL

SEQ ID NO: 136 - protein, 28 aa, SARS-CoV-2 Virus
EIDRLNEVAKNLNESLIDLQELGKYEQY

SEQ ID NO: 137 - protein, 17 aa, SARS-CoV-2 Virus
EVAKNLNESLIDLQELG

SEQ ID NO: 138 - protein, 17 aa, SARS-CoV-2 Virus
GAALQIPFAMQMAYRFN

SEQ ID NO: 139 - protein, 8 aa, SARS-CoV-2 Virus
GAGICASY

SEQ ID NO: 140 - protein, 17 aa, SARS-CoV-2 Virus
AISSVLNDILSRLDKVE

SEQ ID NO: 141 - protein, 8 aa, SARS-CoV-2 Virus
GSFCTQLN

SEQ ID NO: 142 - protein, 17 aa, SARS-CoV-2 Virus
ILSRLDKVEAEVQIDRL

SEQ ID NO: 143 - protein, 8 aa, SARS-CoV-2 Virus
KGIYQTSN

SEQ ID NO: 144 - protein, 8 aa, SARS-CoV-2 Virus
AMQMAYRF

SEQ ID NO: 145 - protein, 17 aa, SARS-CoV-2 Virus
KNHTSPDVDLGDISGIN

SEQ ID NO: 146 - protein, 17 aa, SARS-CoV-2 Virus
MAYRFNGIGVTQNVLYE

SEQ ID NO: 147 - protein, 17 aa, SARS-CoV-2 Virus
AATKMSECVLGQSKRVD

SEQ ID NO: 148 - protein, 17 aa, SARS-CoV-2 Virus
PFAMQMAYRFNGIGVTQ

SEQ ID NO: 149 - protein, 17 aa, SARS-CoV-2 Virus
QALNTLVKQLSSNFGAI

SEQ ID NO: 150 - protein, 17 aa, SARS-CoV-2 Virus
QLIRAAEIRASANLAAT

SEQ ID NO: 151 - protein, 6 aa, SARS-CoV-2 Virus
QQFGRD

SEQ ID NO: 152 - protein, 17 aa, SARS-CoV-2 Virus
RASANLAATKMSECVLG

SEQ ID NO: 153 - protein, 17 aa, SARS-CoV-2 Virus
RLITGRLQSLQTYVTQQ

SEQ ID NO: 154 - protein, 28 aa, SARS-CoV-2 Virus
EIDRLNEVAKNLNESLIDLQELGKYEQY

SEQ ID NO: 155 - protein, 17 aa, SARS-CoV-2 Virus
SLQTYVTQQLIRAAEIR

SEQ ID NO: 156 - protein, 17 aa, SARS-CoV-2 Virus
DLGDISGINASVVNIQK

(19) United States
(12) Patent Application Publication
(10) Pub. No.: US 2021/0283201 A1
(43) Pub. Date: Sep. 16, 2021

(54) Systems and Methods for Treating Patients Infected with SARS-COV-2

(71) Applicant: HDL Therapeutics, Inc., Vero Beach, FL (US)

(72) Inventors: Hollis Bryan Brewer, Jr., Potomac, MD (US); Michael M. Matin, Short Hills, NJ (US)

(21) Appl. No.: 17/191,569

(22) Filed: Mar. 3, 2021

Related U.S. Application Data
(60) Provisional application No. 63/071,513, filed on Aug. 28, 2020.
(60) Provisional application No. 63/004,383, filed on Apr. 2, 2020.
(60) Provisional application No. 62/990,818, filed on Mar. 17, 2020.
(60) Provisional application No. 62/986,136, filed on Mar. 6, 2020.

(51) Int. Cl. A61K 35/76; G01N 33/569

(52) U.S. Cl. CPC A61K 35/76; A61K 35/16; G01N 2333/165; G01N 33/56983

(57) ABSTRACT

The present specification describes methods and systems for treating a patient having a SARS-CoV-2 virus load greater than a predefined threshold. A patient is assessed to determine if the patient has the SARS-CoV-2 virus load greater than the predefined threshold. If the patient has the SARS-CoV-2 virus load greater than the predefined threshold, a volume of plasma is removed from the patient. In a plasma delipidation system, the volume of plasma is mixed with an extraction solvent to delipidate at least some of the SARS-CoV-2 viruses in the patient's SARS-CoV-2 virus load, thereby causing one or more modifications to the SARS-CoV-2 viruses. The extraction solvent is removed from the plasma and the plasma is administered with the at least some of the delipidated SARS-CoV-2 viruses to the patient.

### CROSS-REFERENCE

The present application relies on, for priority, U.S. Patent Provisional Application No. 63/071,513, entitled “Systems and Methods for Treating Patients Infected with SARS-COV-2”, and filed on Aug. 8, 2020, U.S. Patent Provisional Application No. 63/004,383, entitled “Systems and Methods for Treating Patients Infected with SARS-COV-2”, and filed on Apr. 2, 2020, U.S. Patent Provisional Application No. 62/990,818, entitled “Systems and Methods for Treating Patients Infected with SARS-COV-2”, and filed on Mar. 17, 2020, and U.S. Patent Provisional Application No. 62/986,136, entitled “Systems and Methods for Treating Patients Infected with SARS-COV-2”, and filed on Mar. 6, 2020.

The above referenced applications are herein incorporated by reference in their entirety.

### FIELD

The present invention generally relates to systems, apparatuses and methods for treating patients infected with SARS-CoV-2 and variants thereof and/or creating a protective immunological response in patients before they are infected with SARS-CoV-2 and variants thereof or after reaching a predefined viral load level of SARS-CoV-2 and variants thereof.

### BACKGROUND

SARS-CoV-2 is the virus responsible for causing COVID-19 in human patients and animals. In addition, multiple SARS-CoV-2 variants are circulating globally. According to the United States Center for Disease Control, the incubation period is estimated at approximately 5 days, with a wider range of 2-14 days being possible. Frequently reported signs and symptoms include fever, cough, fatigue or myalgia, and shortness of breath. Less commonly reported symptoms include sputum production, headache, hemoptysis, and diarrhea. Some patients have experienced gastrointestinal symptoms such as diarrhea and nausea prior to developing fever and lower respiratory tract signs and symptoms. For certain populations, particularly patients who are 60 years old and older, COVID-19 can be fatal, with mortality rates among certain populations being as high as 20%.

In humans, SARS-CoV-2 infection causes acute lung injury (ALI) with rapid progression to acute respiratory distress syndrome (ARDS), leading to full-blown COVID-19 disease. ARDS is recognized as an acute condition characterized by diffuse alveolar injury, bilateral pulmonary infiltrates, and severe hypoxemia in the absence of evidence for cardiogenic pulmonary edema.

Systemic inflammation is commonly observed in patients infected with COVID-19. Systemic inflammation is often reported to be accompanied by a “cytokine storm,” hemostasis alterations, and severe vasculitis. Over-activation of the immune system due to a SARS-CoV-2 infection causes the “cytokine storm”, which is a surge of pro-inflammatory factors, and results in host organ damage, such as lung damage, increased lung endothelial and epithelial permeability, impaired gas exchange and severe respiratory failure with a high mortality rate. There is emerging evidence that suggests that dysregulation of lipid transport may contribute to some of these complications.

Unfortunately, there are no known anti-viral treatments capable of effectively treating patients infected with SARS-CoV-2. Moreover, there are no known vaccines capable of creating a positive, protective immunological response in patients. Accordingly, there is a very large, urgent need for treatment methods to treat patients infected with SARS-CoV-2, to incite a positive, protective immune response in patients infected with SARS-CoV-2 or in individuals before being infected with SARS-CoV-2, to decrease a viral load in patients infected with SARS-CoV-2, to slow or stop viral replication in patients infected with SARS-CoV-2, and/or to cause a SARS-CoV-2 specific immune response in patients infected with SARS-CoV-2 or in individuals before being infected with SARS-CoV-2.

### SUMMARY

The following embodiments and aspects thereof are described and illustrated in conjunction with systems, tools and methods, which are meant to be exemplary and illustrative, not limiting in scope.

In some embodiments, the present specification is directed toward a method of generating a composition for treating a patient having a coronavirus viral load greater than a predefined threshold, comprising: acquiring plasma from the patient; using a plasma delipidation system, mixing the plasma with an extraction solvent to delipidate at least some of the viruses of the coronavirus viral load, wherein the delipidation of the at least some of the viruses of the coronavirus viral load causes one or more modifications in each of the at least some of the viruses of the coronavirus viral load to form delipidated viruses of the coronavirus viral load; removing the extraction solvent from the plasma with the at least some of the delipidated viruses of the coronavirus viral load; and administering the plasma with the at least some of the delipidated viruses of the coronavirus viral load to the patient.

Optionally, a volume of the plasma is at least 0.5 liters.

Optionally, the method includes repeating each step of claim 1 once per day until the coronavirus viral load of the patient is below a second predefined threshold level.

Optionally, the method includes repeating each step of claim 1 once per day, and no more than three times in a week, until the coronavirus viral load of the patient is below a second predefined threshold level.

Optionally, the method includes repeating each step of claim 1 once per day, and no more than three times in a week, until the coronavirus viral load of the patient is below a second predefined threshold level and a level of the delipidated viruses of the coronavirus viral load is above a third predefined threshold level.

Optionally, the method includes repeating each step of claim 1 once per day for a period of 3 to 7 days.

Optionally, in embodiments, the coronavirus is SARS-CoV-2 or variants thereof.

Optionally, the delipidation process is warranted if the patient's viral load of SARS-CoV-2 or variants thereof is equal to or greater than 9×10\hat{ }10 viral copies per liter of plasma.

Optionally, if the patient's viral load of SARS-CoV-2 or variants thereof is less than 9×10\hat{ }10 viral copies per liter of plasma, the viral load may be increased by introducing live or inactivated, but not denatured virus to the extracted plasma prior to subjecting the plasma to the delipidation process.

Optionally, if the patient's viral load of SARS-CoV-2 or variants thereof is less than 9×10\hat{ }10 viral copies per liter of plasma, the plasma delipidation process may be altered to delipidate both the virus and high-density lipoproteins in extracted plasma using at least one solvent mixture.

Optionally, at least one of the one or more modifications comprises exposing or removing lipids from one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, the method of claim 7, wherein at least one of the one or more modifications comprises modifying a three-dimensional conformational configuration of the target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHID A, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises removing lipid covering one or more epitopes positioned adjacent to one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises increasing a surface accessibility of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises changing a three-dimensional conformational structure of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises decreasing a lipid content of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises modifying a physical position one or more of a target set of epitopes relative to another one of the target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHID A, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, at least one of the one or more modifications comprises modifying a receptor binding domain of each of the SARS-CoV-2 viruses by decreasing an amount of lipid surrounding one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

Optionally, the extraction solvent comprises at least one of alcohols, hydrocarbons, amines, ethers, fluoroethers, surfactants, detergents, or combinations thereof.

Optionally, the extraction solvent comprises sevoflurane.

In some embodiments, the present specification discloses a method of treating a patient having a SARS-CoV-2 virus load greater than a predefined threshold, comprising: assessing the patient to determine if the patient has the SARS-CoV-2 virus load greater than the predefined threshold; if the patient has the SARS-CoV-2 virus load greater than the predefined threshold, removing a volume of plasma from the patient; in a plasma delipidation system, mixing the volume of plasma with an extraction solvent to delipidate at least some of the SARS-CoV-2 viruses in the patient's SARS-CoV-2 virus load, wherein the delipidation of the at least some of the SARS-CoV-2 viruses causes one or more modifications in each of the at least some of the SARS-CoV-2 viruses to form delipidated SARS-CoV-2 viruses, wherein said one or more modifications are: exposing one or more of a target set of epitopes of SARS-CoV-2, SARS-CoV-derived linear B cell epitopes that are identical in SARS-CoV-2, SARS-CoV-derived discontinuous B cell epitopes that have at least one site with an identical amino acid to the corresponding site in SARS-CoV-2; or removing lipid from one or more of the target set of epitopes; or modifying a three-dimensional conformational configuration of the target set of epitopes; or removing lipid covering one or more epitopes positioned adjacent to one or more of the target set of epitopes; or increasing a surface accessibility of one or more of the target set of epitopes; or changing a three-dimensional conformational structure of one or more of the target set of epitopes; or decreasing a lipid content of one or more of the target set of epitopes; or modifying a physical position one or more of the target set of epitopes relative to another one of the target set of epitopes; or modifying a receptor binding domain of the SARS-CoV-2 viral particles by decreasing an amount of lipid surrounding one or more of the target set of epitopes; removing the extraction solvent from the plasma with the at least some of the delipidated SARS-CoV-2 viruses; and administering the plasma with the at least some of the delipidated SARS-CoV-2 viruses to the patient.

Optionally, the volume of plasma is at least 0.5 liters.

Optionally, each step is repeated every day until the SARS-CoV-2 virus load of the patient is below a second predefined threshold level.

Optionally, each step is repeated every day until the SARS-CoV-2 virus load of the patient is below a second predefined threshold level and a level of the delipidated SARS-CoV-2 virus is above a third predefined threshold level.

Optionally, each step of is repeated every day for a period of 3 to 7 days.

The aforementioned and other embodiments of the present specification shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 is a schematic representation of a prior art system comprising a plurality of components used in accordance with some embodiments of the present specification to achieve the processes disclosed herein;

FIG. 2 is a flow chart illustrating an exemplary process for separating modified SAR-CoV-2 viruses using the system of FIG. 1, in accordance with some embodiments of the present specification;

FIG. 3A is a schematic representation of the system illustrating the implementation of the process described in FIG. 2, in accordance with some embodiments of the present specification;

FIG. 3B is a schematic representation of the system illustrating the implementation of the process described in FIG. 2, in accordance with some embodiments of the present specification;

FIG. 3C is a schematic representation of the system illustrating the implementation of the process described in FIG. 2, in accordance with some embodiments of the present specification;

FIG. 3D is a schematic representation of the system illustrating the implementation of the process described in FIG. 2, in accordance with some embodiments of the present specification;

FIG. 3E is a schematic representation of the system illustrating the implementation of the process described in FIG. 2, in accordance with some embodiments of the present specification;

FIG. 3F is a schematic representation of the system illustrating the implementation of the process described in FIG. 2, in accordance with some embodiments of the present specification;

FIG. 3G is a schematic representation of the system illustrating the implementation of the process described in FIG. 2, in accordance with some embodiments of the present specification;

FIG. 3H is a schematic representation of the system illustrating the implementation of the process described in FIG. 2, in accordance with some embodiments of the present specification;

FIG. 3I is a schematic representation of the system illustrating the implementation of the process described in FIG. 2, in accordance with some embodiments of the present specification;

FIG. 3J is a schematic representation of the system illustrating the implementation of the process described in FIG. 2, in accordance with some embodiments of the present specification;

FIG. 3K is a schematic representation of the system illustrating the implementation of the process described in FIG. 2, in accordance with some embodiments of the present specification;

FIG. 3L is a schematic representation of the system illustrating the implementation of the process described in FIG. 2, in accordance with some embodiments of the present specification;

FIG. 3M is a schematic representation of the system illustrating the implementation of the process described in FIG. 2, in accordance with some embodiments of the present specification;

FIG. 3N is a schematic representation of the system illustrating the implementation of the process described in FIG. 2, in accordance with some embodiments of the present specification;

FIG. 3O is a schematic representation of the system illustrating the implementation of the process described in FIG. 2, in accordance with some embodiments of the present specification;

FIG. 4 is a table showing the effect on the reduction of lipids resulting from variation in different chemical and mechanical parameters involved in implementing various embodiments in accordance with the present specification;

FIG. 5 is a table listing another exemplary set of variables that affect the delipidation process and outcome;

FIG. 6 is a table providing another exemplary set of variables that may be used for normal plasma and lipemic IV plasma using different solvents and different methods of separation;

FIG. 7 illustrates an exemplary mixing device, in accordance with embodiments described in context of FIG. 3I;

FIG. 8A illustrates a side view of shaker angle brackets that are used to a position mixing device within a system, in accordance with some embodiments of the present specification;

FIG. 8B illustrates another side view of shaker angle brackets that are used to position a mixing device within a system, in accordance with some embodiments of the present specification;

FIG. 8C illustrates a perspective view of shaker angle brackets that are used to position a mixing device within a system, in accordance with some embodiments of the present specification;

FIG. 9 illustrates a preferred process for treating patients infected with a coronavirus; and

FIG. 10 is a flow chart illustrating an exemplary process of treatment of a patient infected by the SARS-CoV-2 virus, using an adjunctive therapy, in accordance with some embodiments of the present specification.

### SEQUENCE LISTING

As per 37 CFR 1.821(c), Applicant herein incorporates by reference, in its entirety, ASCII text file entitled “HDL301_ST25”, created on May 26, 2021, sized 28 KB, and submitted to the United States Patent and Trademark Office via EFS-Web on May 28, 2021. The amino acid sequences listed in the incorporated sequence listing are shown using standard three letter code abbreviations for amino acids, as defined in 37 C.F.R. 1.822. In the incorporated sequence listing:

SEQ ID Nos: 1-16 show the amino acid sequence of several SARS-Cov-2 virus epitopes;

SEQ ID Nos: 17-43 show the amino acid sequence of several SARS-CoV-derived spike (S) and nucleocapsid (N) protein T cell epitopes that are identical in SARS-CoV-2;

SEQ ID Nos: 44-133 show the amino acid sequence of several SARS-CoV-derived linear B cell epitopes that are identical in SARS-CoV-2; and

SEQ ID Nos: 134-156 show the amino acid sequence of several SARS-CoV-derived discontinuous B cell epitopes that have at least one site with an identical amino acid to the corresponding site in SARS-CoV-2.

### DETAILED DESCRIPTION

In some embodiments, the present specification is directed towards systems, apparatuses and methods for removing lipid from SARS-CoV-2 to expose one or more of the following epitopes, increase the surface accessibility of one or more of the following epitopes, change the three-dimensional conformational structure of one or more of the following epitopes, decrease the lipid content of one or more of the following epitopes, modify the physical position one or more of the following epitopes relative to another one of the following epitopes, or modify the receptor binding domain of the SARS-CoV-2 viral particles by decreasing an amount of lipid surrounding one or more of the following epitopes: KYFKNHTSP (SEQ ID NO: 1), TTKR (SEQ ID NO: 2), YYHKNNKSWM (SEQ ID NO: 3), ASTEK (SEQ ID NO: 4), AWNRKR (SEQ ID NO: 5), EQDKNTQ (SEQ ID NO: 6), GTNTSN (SEQ ID NO: 7), KYNENGT (SEQ ID NO: 8), LDSKTQ (SEQ ID NO: 9), PKKS (SEQ ID NO: 10), YQTQTNSPRRAR (SEQ ID NO: 11), TKRT (SEQ ID NO: 12), DEDDSE (SEQ ID NO: 13), GYQPYRVVVL (SEQ ID NO: 14), QPYRVVVLSF (SEQ ID NO: 15), PYRVVVLSF (SEQ ID NO: 16), SARS-CoV-derived T cell epitopes obtained using positive T cell assays that are identical in SARS-CoV-2 (including ILLNKHID (SEQ ID NO: 17), AFFGMSRIGMEVTPSGTW (SEQ ID NO: 18), MEVTPSGTWL (SEQ ID NO: 19), GMSRIGMEV (SEQ ID NO: 20), ILLNKHIDA (SEQ ID NO: 21), ALNTPKDHI (SEQ ID NO: 22), IRQGTDYKHWPQIAQFA (SEQ ID NO: 23), KHWPQIAQFAPSASAFF (SEQ ID NO: 24), LALLLLDRL (SEQ ID NO: 25), LLLDRLNQL (SEQ ID NO: 26), LLNKHIDAYKTFPPTEPK (SEQ ID NO: 27), LQLPQGTTL (SEQ ID NO: 28), AQFAPSASAFFGMSR (SEQ ID NO: 29), AQFAPSASAFFGMSRIGM (SEQ ID NO: 30), RRPQGLPNNTASWFT (SEQ ID NO: 31), YKTFPPTEPKKDKKKK (SEQ ID NO: 32), GAALQIPFAMQMAYRF (SEQ ID NO: 33), MAYRFNGIGVTQNVLY (SEQ ID NO: 34), QLIRAAEIRASANLAATK (SEQ ID NO: 35), FIAGLIAIV (SEQ ID NO: 36), ALNTLVKQL (SEQ ID NO: 37), LITGRLQSL (SEQ ID NO: 38), NLNESLIDL (SEQ ID NO: 39), QALNTLVKQLSSNFGAI (SEQ ID NO: 40), RLNEVAKNL (SEQ ID NO: 41), VLNDILSRL (SEQ ID NO: 42), VVFLHVTYV (SEQ ID NO: 43)); SARS-CoV-derived spike (S) and nucleocapsid (N) protein T cell epitopes (obtained from positive MHC binding assays) that are identical in SARS-CoV-2 (including FIAGLIAIV (SEQ ID NO: 44), GLIAIVMVTI (SEQ ID NO: 45), IITTDNTFV (SEQ ID NO: 46), ALNTLVKQL (SEQ ID NO: 47), LITGRLQSL (SEQ ID NO: 48), LLLQYGSFC (SEQ ID NO: 49), LQYGSFCT (SEQ ID NO: 50), NLNESLIDL (SEQ ID NO: 51), RLDKVEAEV (SEQ ID NO: 52), RLNEVAKNL (SEQ ID NO: 53), RLQSLQTYV (SEQ ID NO: 54), VLNDILSRL (SEQ ID NO: 55), VVFLHVTYV (SEQ ID NO: 56), ILLNKHID (SEQ ID NO: 57), FPRGQGVPI (SEQ ID NO: 58), LLLLDRLNQ (SEQ ID NO: 59), GMSRIGMEV (SEQ ID NO: 60), ILLNKHIDA (SEQ ID NO: 61), ALNTPKDHI (SEQ ID NO: 62), LALLLLDRL (SEQ ID NO: 63), LLLDRLNQL (SEQ ID NO: 64), LLLLDRLNQL (SEQ ID NO: 65), LQLPQGTTL (SEQ ID NO: 66), AQFAPSASA (SEQ ID NO: 67), TTLPKGFYA (SEQ ID NO: 68), VLQLPQGTTL (SEQ ID NO: 69), GYQPYRVVVL (SEQ ID NO: 70), PYRVVVLSF (SEQ ID NO: 71), LSPRWYFYY (SEQ ID NO: 72), DSFKEELDKY (SEQ ID NO: 73), LIDLQELGKY (SEQ ID NO: 74), PYRVVVLSF (SEQ ID NO: 75), GTTLPKGFY (SEQ ID NO: 76), VTPSGTWLTY (SEQ ID NO: 77), GSFCTQLNR (SEQ ID NO: 78), GVVFLHVTY (SEQ ID NO: 79), AQALNTLVK (SEQ ID NO: 80), MTSCCSCLK (SEQ ID NO: 81), ASANLAATK (SEQ ID NO: 82), SLIDLQELGK (SEQ ID NO: 83), SVLNDILSR (SEQ ID NO: 84), TQNVLYENQK (SEQ ID NO: 85), CMTSCCSCLK (SEQ ID NO: 86), VQIDRLITGR (SEQ ID NO: 87), KTFPPTEPK (SEQ ID NO: 88), KTFPPTEPKK (SEQ ID NO: 89), LSPRWYFYY (SEQ ID NO: 90), ASAFFGMSR (SEQ ID NO: 91), ATEGALNTPK (SEQ ID NO: 92), QLPQGTTLPK (SEQ ID NO: 93), QQQGQTVTK (SEQ ID NO: 94), QQQQGQTVTK (SEQ ID NO: 95), SASAFFGMSR (SEQ ID NO: 96), SQASSRSSSR (SEQ ID NO: 97), TPSGTWLTY (SEQ ID NO: 98), FPNITNLCPF (SEQ ID NO: 99), APHGVVFLHV (SEQ ID NO: 100), FPRGQGVPI (SEQ ID NO: 101), APSASAFFGM (SEQ ID NO: 102), GAALQIPFAMQMAYR (SEQ ID NO: 103), GWTFGAGAALQIPFA (SEQ ID NO: 104), IDRLITGRLQSLQTY (SEQ ID NO: 105), ISGINASVVNIQKEI (SEQ ID NO: 106), LDKYFKNHTSPDVDL (SEQ ID NO: 107), LGDISGINASVVNIQ (SEQ ID NO: 108), LGFIAGLIAIVMVTI (SEQ ID NO: 109), LNTLVKQLSSNFGAI (SEQ ID NO: 110), LQDVVNQNAQALNTL (SEQ ID NO: 111), LQSLQTYVTQQLIRA (SEQ ID NO: 112), LQTYVTQQLIRAAEI (SEQ ID NO: 113), AQKFNGLTVLPPLLT (SEQ ID NO: 114), PCSFGGVSVITPGTN (SEQ ID NO: 115), QIPFAMQMAYRFNGI (SEQ ID NO: 116), QQLIRAAEIRASANL (SEQ ID NO: 117), QTYVTQQLIRAAEIR (SEQ ID NO: 118), AYRFNGIGVTQNVLY (SEQ ID NO: 119), SSNFGAISSVLNDIL (SEQ ID NO: 120), TGRLQSLQTYVTQQL (SEQ ID NO: 121), WLGFIAGLIAIVMVT (SEQ ID NO: 122), CVNFNFNGLTGTGVL (SEQ ID NO: 123), DKYFKNHTSPDVDLG (SEQ ID NO: 124), IDAYKTFPPTEPKKD (SEQ ID NO: 125), MSRIGMEVTPSGTWL (SEQ ID NO: 126), NKHIDAYKTFPPTEP (SEQ ID NO: 127), VLQLPQGTTLPKGFY (SEQ ID NO: 128), LQIPFAMQM (SEQ ID NO: 129), RVDFCGKGY (SEQ ID NO: 130), YEQYIKWPWY (SEQ ID NO: 131), GRLQSLQTY (SEQ ID NO: 132), VRFPNITNL (SEQ ID NO: 133)); SARS-CoV-derived linear B cell epitopes from S (23; 20 of which are located in subunit S2) and N (22) proteins that are identical in SARS-CoV-2 (including DVVNQNAQALNTLVKQL (SEQ ID NO: 134); EAEVQIDRLITGRLQSL (SEQ ID NO: 135); EIDRLNEVAKNLNESLIDLQELGKYEQY (SEQ ID NO: 136); EVAKNLNESLIDLQELG (SEQ ID NO: 137); GAALQIPFAMQMAYRFN (SEQ ID NO: 138); GAGICASY (SEQ ID NO: 139); AISSVLNDILSRLDKVE (SEQ ID NO: 140); GSFCTQLN (SEQ ID NO: 141); ILSRLDKVEAEVQIDRL (SEQ ID NO: 142); KGIYQTSN (SEQ ID NO: 143); AMQMAYRF (SEQ ID NO: 144); KNHTSPDVDLGDISGIN (SEQ ID NO: 145); MAYRFNGIGVTQNVLYE (SEQ ID NO: 146); AATKMSECVLGQSKRVD (SEQ ID NO: 147); PFAMQMAYRFNGIGVTQ (SEQ ID NO: 148); QALNTLVKQLSSNFGAI (SEQ ID NO: 149); QLIRAAEIRASANLAAT (SEQ ID NO: 150); QQFGRD (SEQ ID NO: 151); RASANLAATKMSECVLG (SEQ ID NO: 152); RLITGRLQSLQTYVTQQ (SEQ ID NO: 153); EIDRLNEVAKNLNESLIDLQELGKYEQY (SEQ ID NO: 154); SLQTYVTQQLIRAAEIR (SEQ ID NO: 155); DLGDISGINASVVNIQK (SEQ ID NO: 156)), SARS-CoV-derived discontinuous B cell epitopes (and associated known antibodies [39-41]) that have at least one site with an identical amino acid to the corresponding site in SARS-CoV-2 (including epitopes with IEDB ID 910052, 77444, and 77442); and 7D10 epitope. The delipidation process removes lipids in and around the epitopes, resulting in either a conformational change in the epitopes, epitopes adjacent to the listed epitopes to be exposed, and/or a function of the epitopes to be modified.

In some embodiments, the present specification is directed towards systems, apparatuses and methods for removing lipid from SARS-CoV-2 and/or variants thereof, including, but not limited to D614G mutation; Cluster 5; B.1.1.7 lineage (also known as 20I/501Y.V1 Variant of Concern (VOC) 202012/01), which has a mutation in the receptor binding domain (RBD) of the spike protein at position 501, where the amino acid asparagine (N) has been replaced with tyrosine (Y); B.1.1.207; B.1.351 lineage (a.k.a. 20H/501Y.V2), which has multiple mutations in the spike protein, including K417N, E484K, N501Y; P.1 lineage (a.k.a. 20J/501Y.V3), which is a branch off the B.1.1.28, where the P.1 lineage contains three mutations in the spike protein receptor binding domain: K417T, E484K, and N501Y; B.1.427; B.1.525; B.1.429 (CAL.20C); and any other variant that may present. Alm and Broberg et al. discuss various SARS-CoV-2 variants, identified from January 2020 to June 2020 (Alm Erik, Broberg Eeva K, Connor Thomas, Hodcroft Emma B, Komissarov Andrey B, Maurer-Stroh Sebastian, Melidou Angeliki, Neher Richard A, O'Toole Aine, Pereyaslov Dmitriy, The WHO European Region sequencing laboratories and GISAID EpiCoV group. Geographical and temporal distribution of SARS-CoV-clades in the WHO European Region, January to June 2020. Euro Surveill. 2020; 25(32):pii=2001410. https://doi.org/10.2807/1560-7917.ES.2020.25.32.2001410), which is herein incorporated by reference in its entirety. It should be understood by those of ordinary skill in the art that the use of the term SARS-CoV-2 throughout the specification includes all variants thereof.

In some embodiments, the methods and systems of the present specification also provides adjunctive therapeutic approaches that modulate lipids and lipoproteins. COVID-19 may cause changes in the quantity and composition of high-density lipoprotein (HDL), which significantly decreases the anti-inflammatory and anti-oxidative functions of HDL and may contribute to pulmonary inflammation. Also, lipoproteins with oxidized phospholipids and fatty acids may lead to virus-associated organ damage via over-activation of innate immune scavenger receptors. Embodiments of the present specification restore lipoprotein function via ApoA-I raising agents or via blocking relevant scavenger receptors with neutralizing antibodies, which both serve as adjunctive therapies in the treatment of COVID-19. Embodiments of the present specification also use omega-3 fatty acids transported by lipoproteins to generate specialized pro-resolving mediators. In embodiments, when coupled with anti-inflammatory drugs, the therapies presented in the present specification may decrease inflammation and thrombotic complications associated with COVID-19.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

The term “fluid” shall mean any fluid containing an infectious organism, including but not limited to, a biological fluid obtained from an organism such as an animal or human. Preferred infectious organisms treated with the method of the present invention are viruses, particularly coronaviruses and more particularly SARS-CoV-2. Such biological fluids obtained from an organism include but are not limited to blood, plasma, serum, cerebrospinal fluid, lymphatic fluid, peritoneal fluid, follicular fluid, amniotic fluid, pleural fluid, pericardial fluid, reproductive fluids and any other fluid contained within the organism. Other fluids may include laboratory samples containing infectious organisms suspended in any chosen fluid. Other fluids include cell culture reagents, many of which include biological compounds such as fluids obtained from living organisms, including but not limited to “normal serum” obtained from various animals and used as growth medium in cell and tissue culture applications.

By the terms “first solvent” or “first organic solvent” “or first extraction solvent” are meant a solvent, comprising one or more solvents, used to facilitate extraction of lipid from a fluid or from a lipid-containing biological organism in the fluid. This solvent will enter the fluid and remain in the fluid until being removed. Suitable first extraction solvents include solvents that extract or dissolve lipid, including but not limited to alcohols, hydrocarbons, amines, ethers, fluoroethers (including but not limited to fluoromethyl hexafluoroisopropyl ether (Sevoflurane)), surfactants, detergents, and combinations thereof. First extraction solvents may be combinations such as the following: 1) an alcohol and an ether; 2) an alcohol and a fluoroether; 3) an alcohol and a surfactant, 4) an ether and a surfactant; or 5) an alcohol, an ether and a surfactant. First extraction solvents include, but are not limited to n-butanol, di-isopropyl ether (DIPE), fluoroether such as sevoflurane, surfactants such as TRITON X-100™ (polyethylene glycol p-(1,1,3,3-tetramethylbutyl)-phenyl ether or polyoxyethylene octyl phenyl ether) or Tween 20™ (PEG(20)sorbitan monolaurate), diethyl ether, and combinations thereof.

The term “second extraction solvent” is defined as one or more solvents that may be employed to facilitate the removal of a portion of the first extraction solvent. Suitable second extraction solvents include any solvent that facilitates removal of the first extraction solvent from the fluid. Second extraction solvents include any solvent that facilitates removal of the first extraction solvent including but not limited to ethers, alcohols, hydrocarbons, amines, and combinations thereof. Preferred second extraction solvents include diethyl ether and di-isopropyl ether, which facilitate the removal of alcohols, such as n-butanol, from the fluid. The term “de-emulsifying agent” is a second extraction solvent that assists in the removal of the first solvent which may be present in an emulsion in an aqueous layer.

The term “delipidation” refers to the process of removing at least a portion of a total concentration of lipids in a fluid or in a virus.

The terms “pharmaceutically acceptable carrier” or “pharmaceutically acceptable vehicle” are used herein to mean any liquid including but not limited to water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner.

The term “patient” refers to animals and humans.

The term “patient specific antigen” refers to an antigen that is capable of inducing a patient specific immune response when introduced into that patient. Such patient specific antigens may be viral antigens. A patient specific antigen includes any antigen, for example a viral antigen, that has been modified or influenced within the patient.

### Preferred Treatment Process

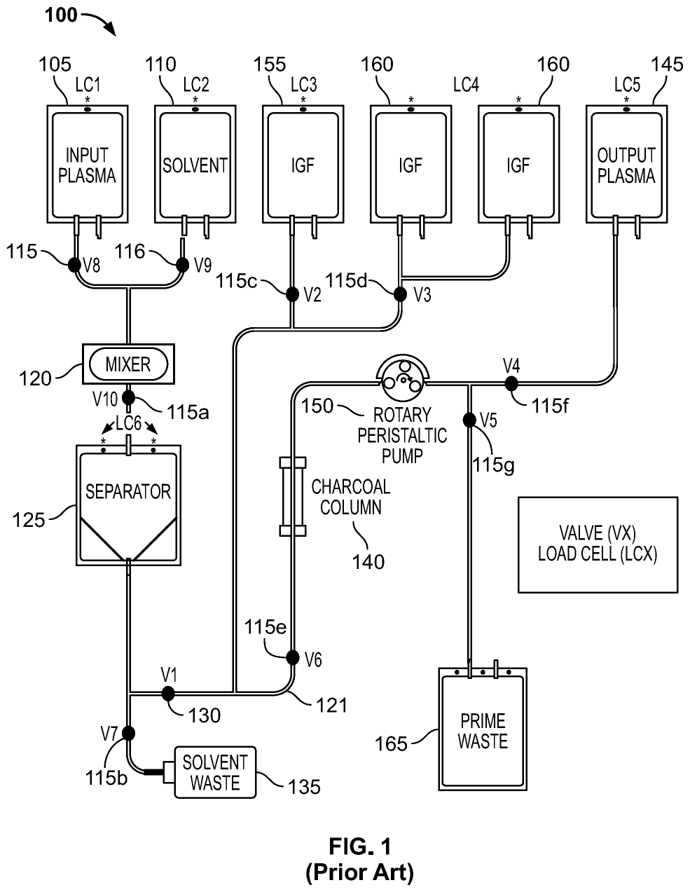

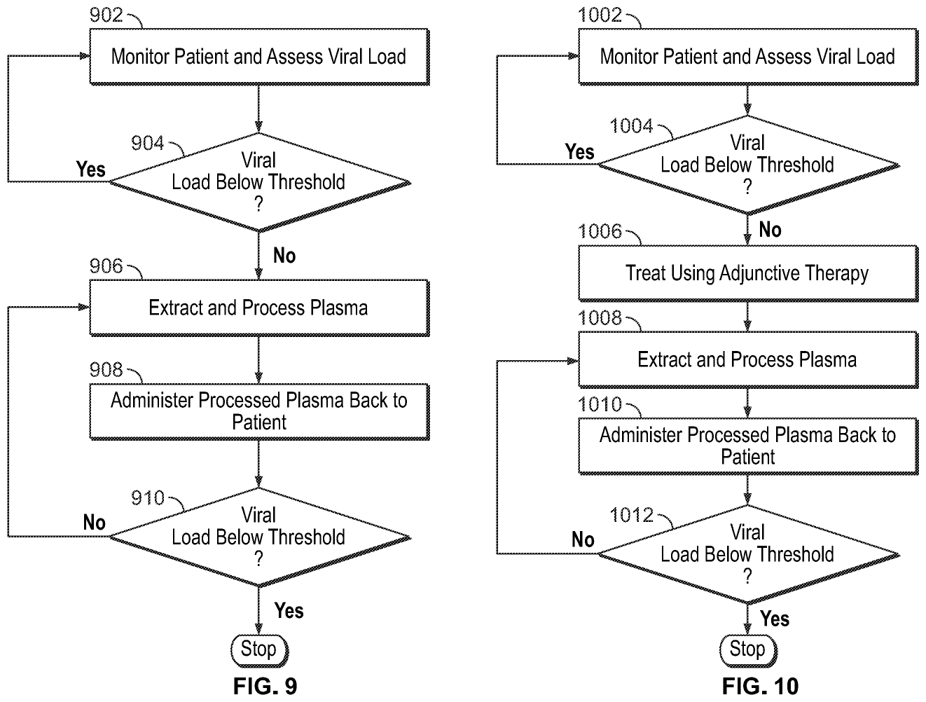

FIG. 9 is a flow chart illustrating an exemplary process of treatment of a patient infected by the SARS-CoV-2 virus, in accordance with some embodiments of the present specification. At step 902, the patient is tested and diagnosed to be positive for the SARS-CoV-2 virus. A viral load of the patient diagnosed to be positive, is assessed using methods known to persons of ordinary skill in the art. At step 904, it is determined whether the assessed viral load is above or below a threshold. If below the threshold, the patient is continually or periodically monitored to observe the viral load. In some optional embodiments, the viral load may be artificially increased to arrive at the predetermine threshold. If and when the viral load meets or exceeds the threshold, treatment is initiated in accordance with the various embodiments of the present specification. In some embodiments, the patient is administered with interferon beta at the beginning of the treatment and as needed, such as for example with an injection of 100 ml to 500 ml, or more preferably 200-300 ml. At step 906, a predefined amount of plasma is extracted from the patient. In an exemplary case, on a first day of treatment, a liter of plasma is extracted from the patient. It should be appreciated that the amount of plasma extracted is based on a maximum amount of plasma an ill patient can manage losing and may range from 0.1 liters to 1.5 liters in a day. The extracted plasma is processed in accordance with the delipidation treatment procedure and the delipidation apparatus, described in context of FIGS. 1 to 8B. At step 908, the processed plasma is administered back to the patient. In an exemplary case, the processed plasma is administered back to the patient within a few hours and on the same day of extracting the plasma. In some embodiments, the delipidation process requires about 3 to 4 hours, after which the processed plasma is administered back to the patient. At step 910, the patient is monitored again to determine the viral load. If the viral load is determined to be at or above the first predefined threshold level, the procedure of the first day is repeated on the second day, and on subsequent days, until the viral load is brought below a second predefined threshold level. In a preferred embodiment, the procedure is repeated once per day, and no more than three times in a week, until the coronavirus viral load is below a second predefined threshold level. In another preferred embodiment, the procedure is repeated once per day, and no more than three times in a week, until the coronavirus viral load is below a second predefined threshold level and a level of the delipidated viruses of the coronavirus viral load is above a third predefined threshold level. In an embodiment, if the viral load is determined to be at or above the threshold, the procedure of the first day is repeated on the second day, and on subsequent days, until the viral load is brought below the threshold. In some cases, the treatment procedure of FIG. 9 is repeated over a duration of three to seven days, comprising extracting about three to seven liters of plasma from the patient. In some embodiments, the treatment method of FIG. 9 is performed without determining the viral load for a duration of three to seven days, sometimes up to 10 days. Each time the plasma is processed, the viral load decreases, and the patient is administered with inactive, modified, SARS-CoV-2 virus, thereby allowing the patient to create antibodies and fight the disease.

In one embodiment, the delipidation process is optimized to reduce lipid content in the replication-transcription complex (RTC) formed in double-membrane vesicles of SARS-CoV-2. Specifically, the solvent combination, amount of solvent used, extent of mixing time, and other processing steps disclosed herein are each selected to decrease the lipid content of the replication-transcription complex (RTC) formed in double-membrane vesicles of SARS-CoV-2 by a minimum of 5%, preferably any increment within a range 5% to 100%.

Adjunctive Therapies

In addition to the delipidation process described above and below, which, in one embodiment, is used to generate a modified SAR-CoV-2 particle that has immunogenic properties, and optionally combined with a pharmaceutically acceptable carrier to make a composition comprising a vaccine, the modified SARS-CoV2 viral particle can be combined with adjuvant pharmacological therapies to suppress the “cytokine storm”, thus protecting against lung damage. In some embodiments, the cytokine storm is prevented by blocking individual inflammatory cytokines with anti-IL-1 or anti-IL-6 therapies. The blockade of a single cytokine, however, may be insufficient to suppress the “cytokine storm,” which involves multiple cytokines and redundant systems maintaining the inflammatory response.

High-density lipoprotein-associated apolipoproteins, such as apolipoprotein A-I (ApoA-I) and apolipoprotein M (ApoM), interact with lipid rafts on cellular membranes that are enriched in immune cell receptors, such as Toll-like receptors on macrophages and T-cell receptors and modulate the immune responses. There is known to be a causal inference for an inverse relationship between HDL-C, but not LDL-C or triglycerides, and risk of an infectious disease hospitalization. In the context of COVID-19, it has been reported that low levels of total cholesterol (TC), HDL-C and LDL-C are associated with disease severity and mortality.

It has been observed that inflammation alters HDL apolipoprotein composition. Inflammation alters hepatic apolipoprotein gene expression and promotes binding of the pro-inflammatory serum amyloid protein A (SAA) which, in turn, displaces and decreases ApoA-I levels in HDL. Moreover, in the setting of acute inflammation, decreased plasma levels of lecithin cholesterol acyltransferase (LCAT) may also alter HDL function and further deteriorate the inflammatory response. Treatment of inflammation-altered HDL with LCAT ex-vivo reduces HDL-bound SAA, while increasing HDL-bound ApoA-I, and HDL function. It has been recently shown that SAA plasma levels are dynamically elevated with COVID-19 disease severity and SAA has been proposed as a biomarker for evaluating the severity and prognosis of COVID-19. Treatment of inflammation-altered HDL with LCAT ex-vivo, significantly decreases HDL-bound paraoxonase 1 (PON1), an antioxidant enzyme present in HDL. PON1 is also inactivated under inflammation-induced oxidative stress, which further compromises HDL function. These findings suggest that HDL protein composition and function are altered in COVID-19 patients, raising the possibility that interventions, such as the LCAT treatment, may improve HDL function and reduce disease burden. Therefore, embodiments of the present specification combine the LCAT treatment with the delipidation process, which in one embodiment is used to generate the modified SAR-CoV-2 particle that has immunogenic properties.

Interventions to either improve HDL functionality, such as increasing LCAT activity mentioned above, or replenishment with functional HDL or relevant apolipoproteins, such as ApoA-I mimetic peptides are effective in improving HDL functionality for the management of SARSCoV-2 related complications, and are included within the scope of the embodiments of the present specification.

Besides ApoA-I, ApoE is also found on HDL, as well as ApoB-containing lipoproteins. Complete ApoE deficiency in humans results in increased plasma levels of triglycerides and cholesterol in ApoB-containing lipoproteins, decreased HDL-cholesterol, palmar-tuberoeruptive xanthoma, and premature cardiovascular disease. The ApoE gene is associated with modified lung physiology in humans. The ApoE4 variant has been reported to predict COVID-19 severity. Therefore, embodiments of the present specification enable increase in both ApoE and phospholipid transfer protein (PLTP) activity, which improves the delivery of energy substrates and phospholipids to tissues for sustaining cellular membrane homeostasis in intensive care patients.

Low-density lipoprotein is the main vehicle for transporting cholesterol and phospholipids in the human circulation. During acute inflammation, LDL and its major apolipoprotein, apolipoprotein B (apoB) are oxidized (oxLDL). Lipid hydroperoxides derived from the lipoxygenase pathway, and hydroxy fatty acids derived from arachidonic acid (AA) and linoleic acid (LA) accumulate and some are esterified into cholesterol esters, triacylglycerol, and phospholipids in oxLDL. Oxidized phospholipids (OxPLs) production is increased in the lungs of virus infected humans and animals and oxPL induces macrophage cytokine production and acute lung inflammation. The oxLDL scavenger receptor lectin-like oxLDL receptor (LOX-1), expressed in endothelial cells, macrophages, and smooth muscle cells, binds multiple ligands, including oxLDL, oxHDL, C-reactive protein, and advanced glycated end products. Because of its binding and response to dysfunctional lipids, such as oxLDL and oxHDL, LOX-1 may be a key mediator of CVD by inducing inflammation-triggered atheroma growth and eventually plaque erosion and rupture. The association of LOX-1 activation and acute inflammatory conditions raises the possibility that LOX-1 is also activated and may contribute to COVID-19 complications. Recent data has also suggested that SARS-CoV-2 may cause a pediatric multisystem inflammatory syndrome reminiscent of Kawasaki Disease (KD) in children. There has been evidence for LOX-1 overactivation in KD patients. Therefore, embodiments of the present specification enable blocking of LOX-1 plays an important role in COVID-19 complications and is another target for therapy, in accordance with the embodiments of the present specification.

The continuous inflammatory response driven by the “cytokine storm,” which includes release of the pro-inflammatory cytokines (TNF-α, IL-6, IL-8, and IL-10) and lymphopenia, are considered to be one of the main cause of life-threatening complications in SARS-CoV-2 patients. The direct effect of the released cytokines and chemokines results in massive cell death that provokes a cascade of biological reactions, including production of macrophage-derived eicosanoids that further potentiate inflammation. Inflammation resolution is an active process mediated by specific molecules collectively referred to as specialized proresolving mediators (SPMs). In embodiments of the present specification, patients are treated with omega-3 polyunsaturated fatty acids (PUFAs), which are transported in plasma on lipoproteins, and serve as precursors to SPMs production by macrophages and neutrophils. The PUFAs are provided in the form of a supplementary diet. In some embodiments, supplementation with omega-3 fatty acids and aspirin is used to significantly reduce pro-inflammatory eicosanoids even further.

In embodiments, omega-3 fatty acids are used in several therapeutic treatments using EPA monotherapy or as a supplement mixture containing EPA, gamma-linolenic acid, and antioxidants in COVID-19 patients.

In one embodiment, nonsteroidal anti-inflammatory drugs (NSAIDs) are used as cyclooxygenase-1 (COX-1) and COX-2 inhibitors, in the form of an adjunctive therapy to the delipidation process of the present specification. In an embodiment, the NSAID therapy uses a combination of Aspirin (acetylsalicylic acid), Losartan, Simvastatin, and Vitamin D. In another embodiment, the NSAID therapy uses Naproxen.

In one embodiment, corticosteroids, which are known to be using multifactorial mechanism of action, are used as an adjunctive therapy to the treatment provided in accordance with embodiments of the present specification.

In one embodiment, statins are used as HMG-CoA reductase inhibitors, in the form of an adjunctive therapy to the delipidation process of the present specification. In an embodiment, the statin therapy uses one or more of Ulinastatins and Atorvastatin.

In one embodiment, Sitagliptin is used as a Dipeptidyl peptidase-4 (DPP-4) inhibitor, in the form of an adjunctive therapy to the delipidation process of the present specification.

In yet another embodiment, colchicine is used for inhibition of microtubule polymerization, in the form of an adjunctive therapy to the delipidation process of the present specification.

The “cytokine storm” underlying COVID-19 produces immune-mediated inflammatory dyslipoproteinemia, leading to low HDL-C and LDL-C levels, elevated triglycerides, increased lipoprotein oxidation, low ApoE levels, and impaired inflammation resolution due to decreased SPMs biosynthesis. These lipid abnormalities might be modified by pharmacological agents, as described above, that increase ApoA-I and HDL plasma levels. Raising HDL also restores lipid transport function, along with improving the antioxidant properties of HDL. While the above embodiments, describe a few adjunctive therapies to the treatment of COVID-19 in accordance with the embodiments of the present specification, other HDL-raising pharmacological compounds can also be used. These compounds include and are not limited to fibrates, CETP-inhibitors, recombinant LCAT, and small molecules that upregulate ApoA-I production.

FIG. 10 is a flow chart illustrating an exemplary method for the treatment of a patient infected by the SARS-CoV-2 virus, using an adjunctive therapy, in accordance with some embodiments of the present specification. At step 1002, the patient is tested and diagnosed as SARS-CoV-2 positive. A viral load of the patient diagnosed as COVID positive is assessed using methods known to persons of ordinary skill in the art. At step 1004, it is determined whether the assessed viral load is above or below a predetermined threshold. If below the threshold, the patient is continually or periodically monitored to observe the viral load. If and when the viral load exceeds the threshold, treatment is initiated in accordance with the various embodiments of the present specification. In some embodiments, interferon beta is administered to the patient at the beginning of the treatment and as needed, such as for example with an injection of 100 ml to 500 ml, or more preferably 200-300 ml. At step 1006, an adjunctive therapy is administered to the patient that further increase ApoA-I and HDL plasma levels, thereby supplementing the remaining treatment. The adjunctive therapy is in accordance with at least one of the adjunctive therapies described above and including one of: increase in LCAT activity; blockade of lectin-like oxidized low-density lipoprotein receptor-1 (LOX-1); NSAID (Aspirin, Aspirin plus Losartan plus Simvastatin, Aspirin plus Vitamin D); Naproxen; Corticosteroids (Multifactorial); Statins (HMG-CoA reductase inhibitor); Omega-3 PUFAs (Multifactorial, specialized proresolving mediators, EPA, EPA plus gamma-linolenic acid and anti-oxidants, Omega-3 plus aspirin to reduce pro-inflammatory eicosanoids); Sitagliptin (Dipeptidyl peptidase-4 (dpp-4) inhibitor); and Colchicine (inhibition of microtube polymerization). In embodiments, step 1006 is performed parallel to the remaining steps. At step 1008, a predefined amount of plasma is extracted from the patient. In an exemplary case, on a first day of treatment, a liter of plasma is extracted from the patient. It should be appreciated that the amount of plasma extracted is based on a maximum amount of plasma an ill patient can manage losing and may range from 0.1 liters to 1.5 liters in a day. The extracted plasma is processed in accordance with the delipidation treatment procedure and the delipidation apparatus, described in context of FIGS. 1 to 8B. At step 1010, the processed plasma is administered back to the patient. In the exemplary case, the processed plasma is administered back to the patient within a few hours and on the same day of extracting the plasma. In some embodiments, the delipidation process requires about 3 to 4 hours, after which the processed plasma is administered back to the patient.

At step 1012, the patient is monitored again to determine the viral load. If the viral load is determined to be at or above the first predefined threshold level, the procedure of the first day is repeated on the second day, and on subsequent days, until the viral load is brought below a second predefined threshold level. In a preferred embodiment, the procedure is repeated once per day, and no more than three times in a week, until the coronavirus viral load is below a second predefined threshold level. In another preferred embodiment, the procedure is repeated once per day, and no more than three times in a week, until the coronavirus viral load is below a second predefined threshold level and a level of the delipidated viruses of the coronavirus viral load is above a third predefined threshold level. In an embodiment, if the viral load is determined to be at or above the threshold, the procedure of the first day is repeated on the second day, and on subsequent days, until the viral load is brought below the threshold. In some exemplary cases, the treatment procedure of FIG. 10 is repeated over a duration of three to seven days, comprising extracting about three to seven liters of plasma from the patient. In some embodiments, the treatment method of FIG. 10 is performed without determining the viral load for a duration of three to seven days, sometimes up to 10 days. Each time the plasma is processed, the viral load is decreased and the patient is administered with inactive, modified, SARS-CoV-2 virus, thereby allowing the patient to create antibodies and fight the disease, while also treating lung inflammation.

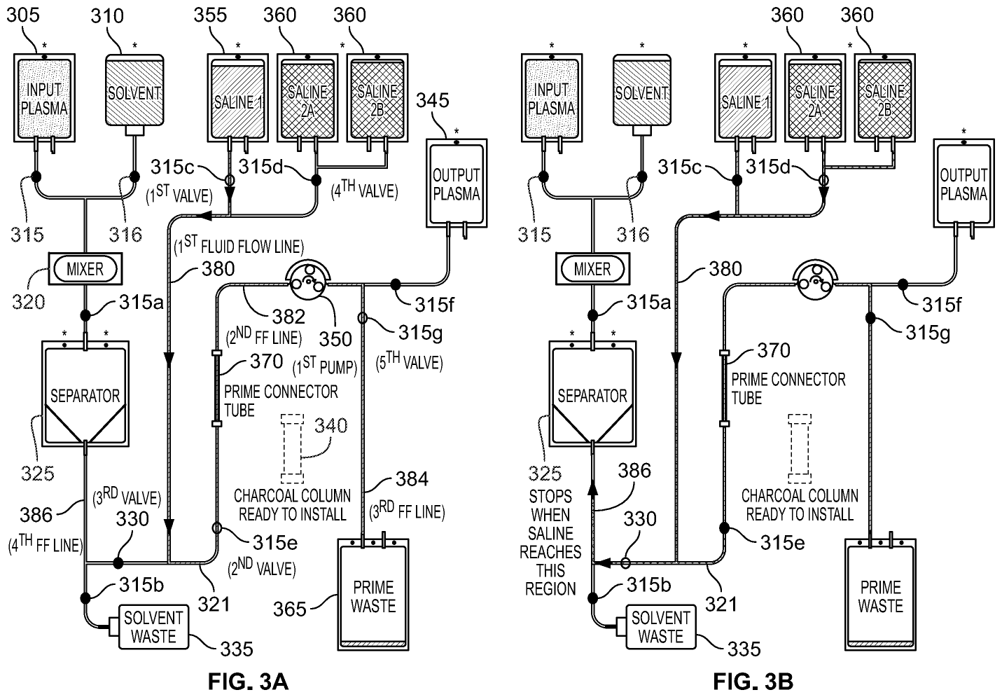

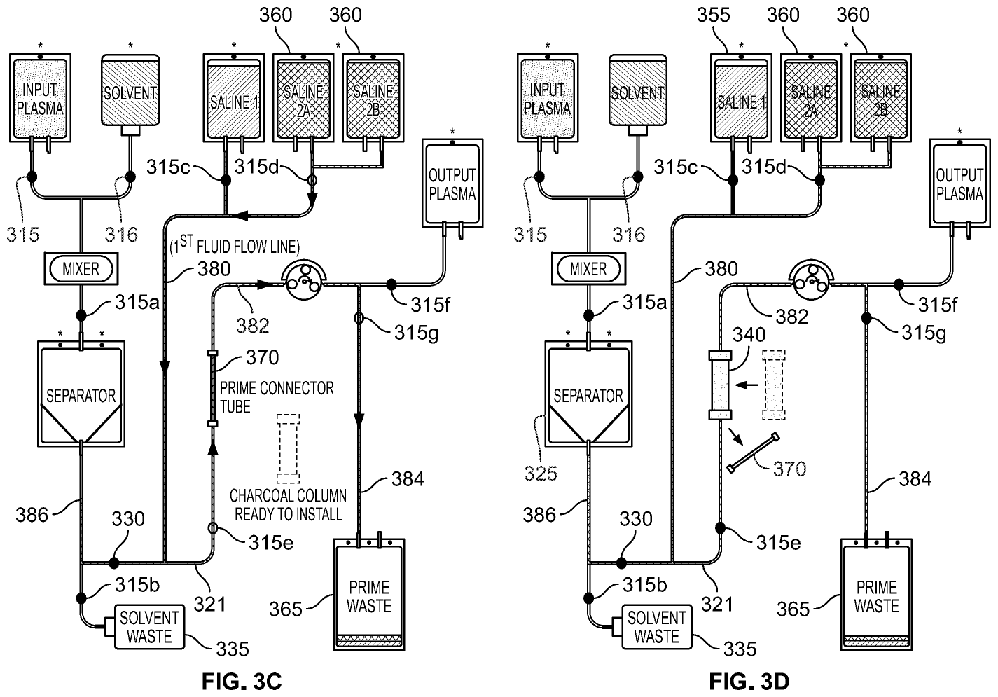

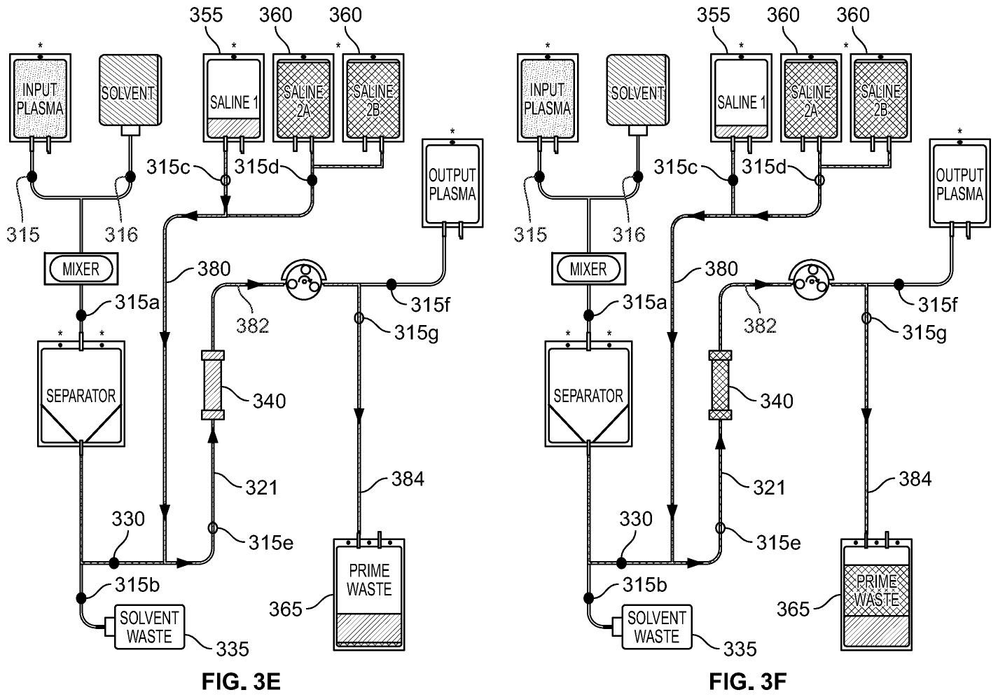

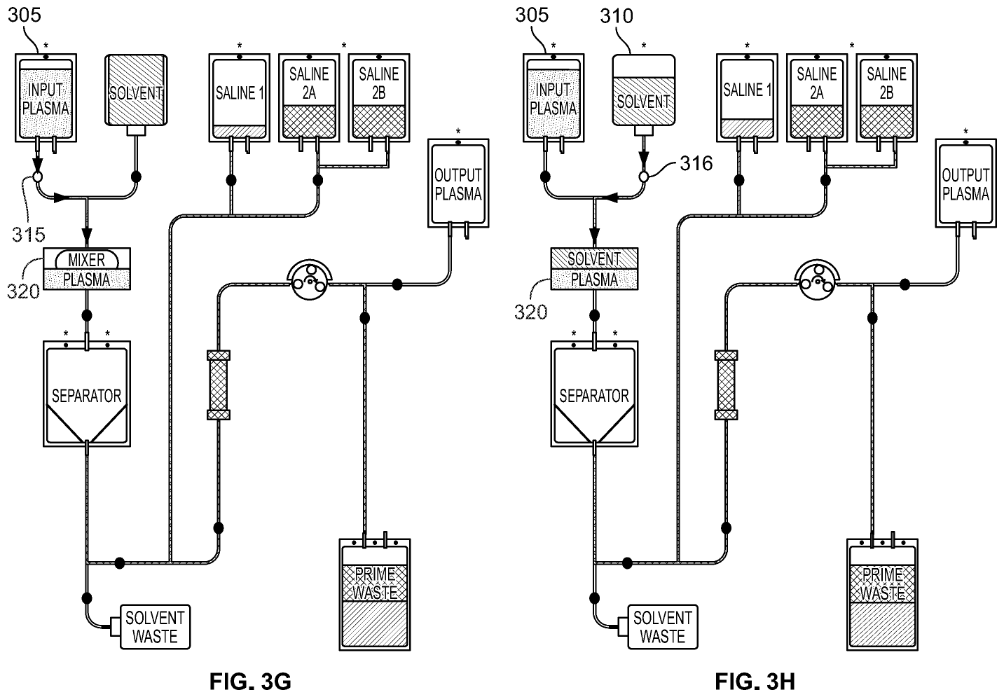

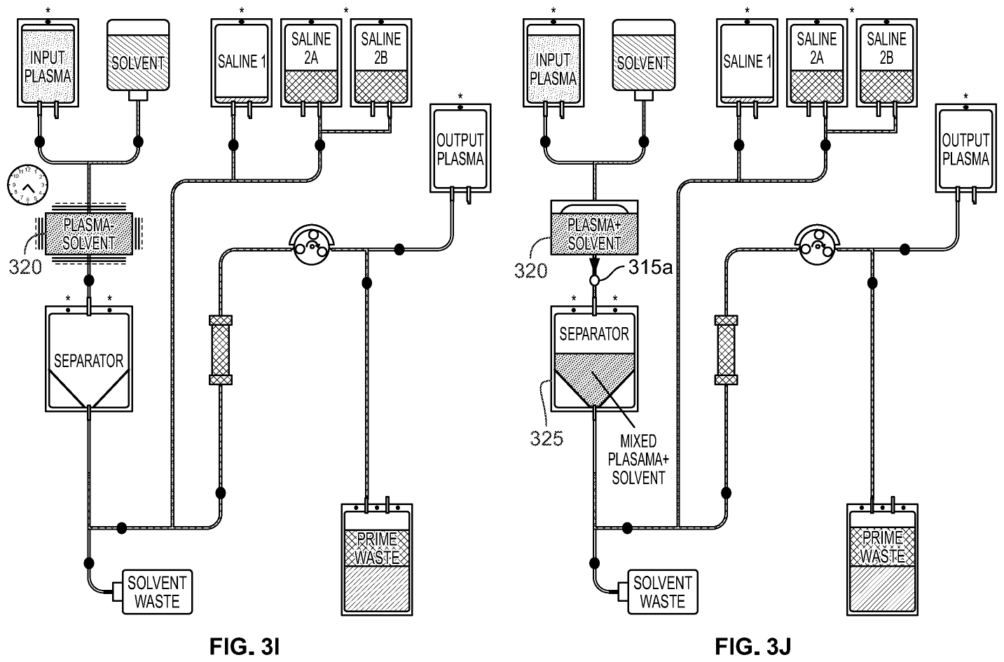

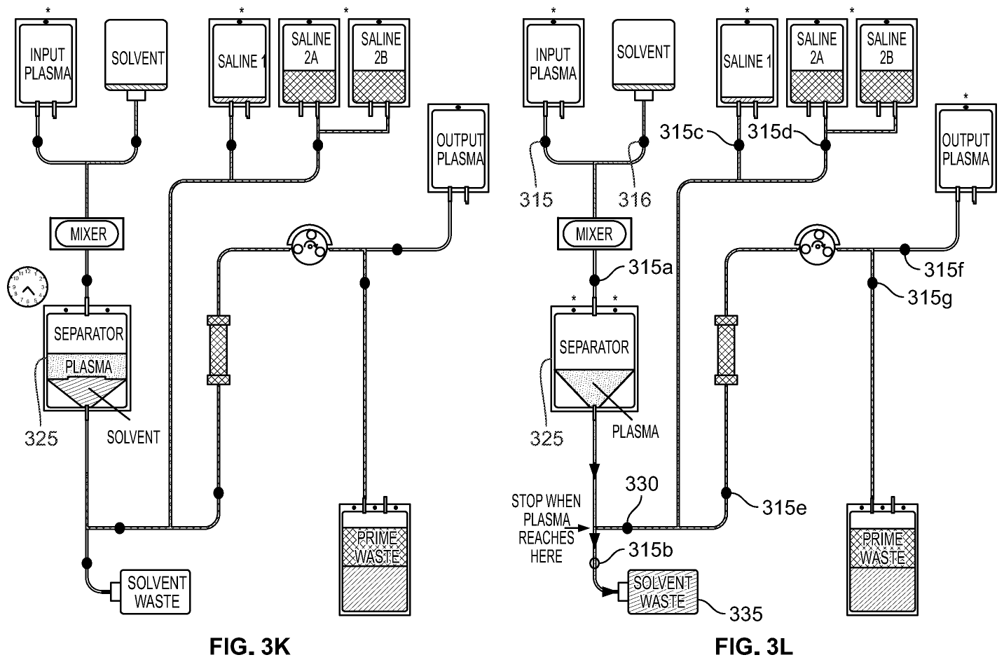

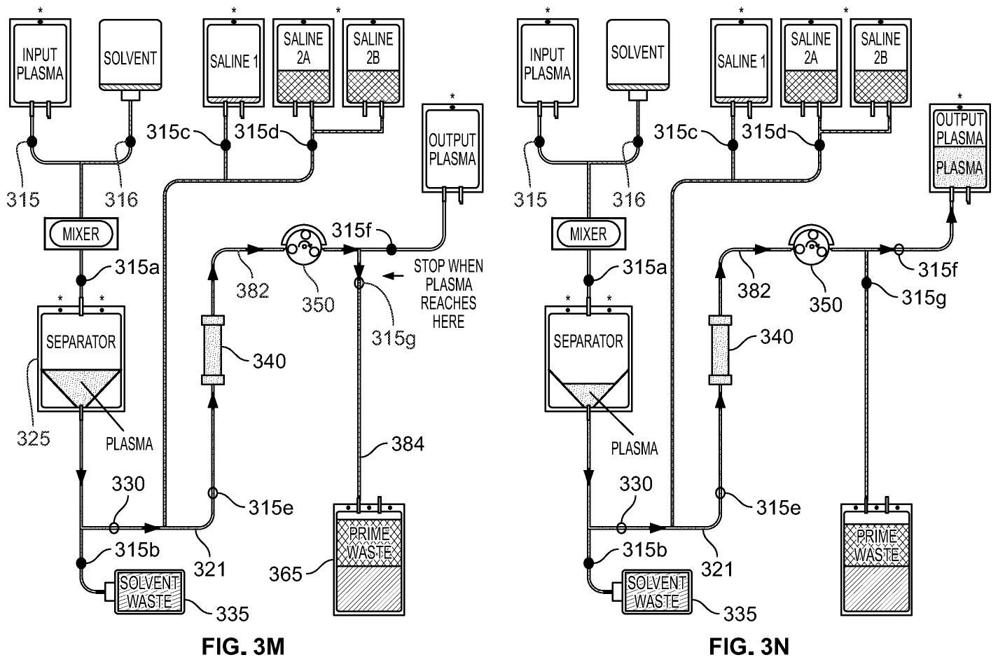

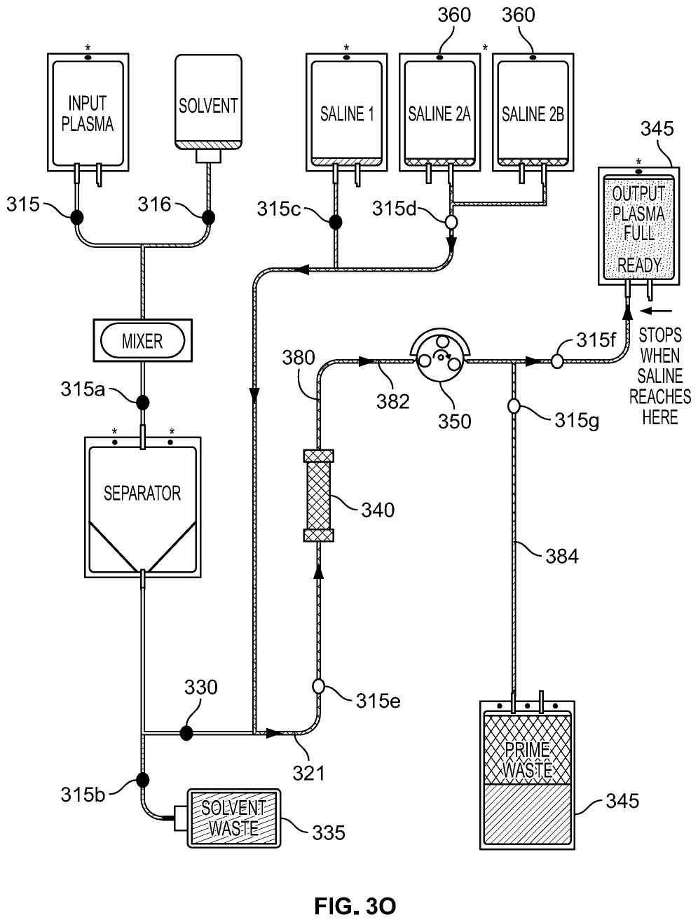

The apparatus of FIG. 1 and methods and apparatus of FIGS. 3A to 3O, described subsequently, may be used for the treatment in accordance with the processes of FIGS. 9 and 10. In some embodiments, the apparatus of FIG. 1 supports two patients for the treatment period, which may span across three to ten days. If the apparatus is used in shifts for 24 hours in day, then a single apparatus could support treatment of four patients. Towards the end of the treatment, the patient has an active, unmodified SARS-CoV-2 viral load of less than a predefined threshold and an inactivated, modified SARS-CoV-2 load of more than a predefined threshold, which is adapted to incite a positive, protective immunological response.

Patients with Insufficient Viral Loads in Plasma

In order to effectively delipidate SARS-CoV-2 in a manner that invokes an immune response in a patient, the patient's plasma must have a sufficiently high viral load such that, when delipidated, leaves enough viral protein to actually invoke an antibody and cellular immune response in the patient. However, in some viral infections, including SARS-CoV-2, the virus is not found in appreciable concentrations in a patient's plasma. In particular, it has been reported that certain SARS-CoV-1 only has 5.89×10⁸ virus copies per liter of plasma, or less and SARS-CoV-2 manifests at an even lower concentration. Unfortunately, for the prior art approaches, plasma viral concentrations need to be an order of magnitude larger to yield any therapeutic benefit. For example, to obtain at least 0.1 micrograms of SARS-CoV-2 in one liter of plasma, there would need to be at least 9×10¹⁰ copies of the virus per liter of extracted plasma. Delipidating a lower number of viral particles will yield an insufficiently low amount of delipidated viral protein and will not result in an effective immune response in the patient.

To treat patients suffering from a virus, such as SARS-CoV-2 and/or its variants, which does not appreciably manifest in plasma, a supplementary treatment process is required. For purposes of this invention, such a patient would have an average viral load, during the course of the patient's illness and prior to the patient's death, that is less than 9×10¹⁰ viral copies per liter of plasma. When faced with a patient having such a low viral load, the presently disclosed treatment methods and systems may be supplemented by artificially increasing the viral load in extracted plasma or by tuning the plasma delipidation process to delipidate both virus, which typically requires less aggressive solvents, and high-density lipoproteins, which typically requires more aggressive solvents, in extracted plasma.

Viral loads may be artificially increased in extracted plasma by taking the extracted plasma and introducing live virus, or inactivated but not denatured virus, that has been dissolved or mixed into saline or some other biocompatible fluid. In one embodiment, pelletized live virus, or inactivated but not denatured virus, is dissolved into saline. The virus mixture is placed into an IV bag and placed in controlled fluid communication with the extracted plasma. A sufficient amount of the virus mixture is allowed to flow into, or mix with, the extracted plasma in the IV bag to achieve an increase in viral load up to, or beyond, 9×10¹⁰ copies of the SARS-CoV-2 virus per liter of extracted plasma. The plasma with increased viral load is then subjected to delipidation as disclosed herein.

Alternatively or concurrently, a solvent system is used that would concurrently delipidate both the low viral load in plasma and the high density lipoproteins in plasma. Prior art delipidation methods have focused on delipidating either virus or high density lipoproteins. By using a solvent system that does both, the benefits of high-density lipoprotein delipidation for treating viral infections, particularly the cytokine storm or inflammatory effects of SARS-CoV-2 infections, can be achieved. More specifically, in patients suffering from a SARS-CoV-2 infection, it would be beneficial to provide anti-inflammatory therapy by extracting plasma from a patient, applying a solvent system that delipidates both high density lipoprotein and SARS-CoV-2 in plasma without denaturing, killing, or otherwise destroying the underlying particle (high density lipoprotein and SARS-CoV-2), and then reinfusing the delipidated plasma back into the patient. Such a process concurrently induces an antibody response, cellular immune response, and beneficial anti-inflammatory response.

### Modified Viral Particle

The above-described process results in the creation of a modified SARS-CoV-2 virus. The modified SARS-CoV-2 viral particles have lower levels of cholesterol and are immunogenic and have exposed epitopes that are not usually presented to the immune system by untreated virus. The modified viral particle has a lower lipid content in the envelope, displays modified proteins, reduced infectivity and is immunogenic. The delipidation methods provided herein do not lead to destruction of the viral envelope of the modified, partially delipidated immunogenic viral particles. A significant proportion of the viral envelopes are present following the partial delipidation. Thus, some embodiments of the partial delipidation methods provided herein result in partially delipidated particles comprising viral envelopes, including envelope proteins.

In one embodiment, the treatment process described above, and delipidation processes described herein, result in the exposure of one or more of the following epitopes, increases in the surface accessibility of one or more of the following epitopes, changes in the three-dimensional conformational structure of one or more of the following epitopes, decreases in the lipid content of one or more of the following epitopes, modifications of the physical position one or more of the following epitopes relative to another one of the following epitopes, or modifications of the receptor binding domain of the SARS-CoV-2 viral particles by decreasing an amount of lipid surrounding one or more of the following epitopes: KYFKNHTSP (SEQ ID NO: 1), TTKR (SEQ ID NO: 2), YYHKNNKSWM (SEQ ID NO: 3), ASTEK (SEQ ID NO: 4), AWNRKR (SEQ ID NO: 5), EQDKNTQ (SEQ ID NO: 6), GTNTSN (SEQ ID NO: 7), KYNENGT (SEQ ID NO: 8), LDSKTQ (SEQ ID NO: 9), PKKS (SEQ ID NO: 10), YQTQTNSPRRAR (SEQ ID NO: 11), TKRT (SEQ ID NO: 12), DEDDSE (SEQ ID NO: 13), GYQPYRVVVL (SEQ ID NO: 14), QPYRVVVLSF (SEQ ID NO: 15), PYRVVVLSF (SEQ ID NO: 16), SARS-CoV-derived T cell epitopes obtained using positive T cell assays that are identical in SARS-CoV-2 (including ILLNKHID (SEQ ID NO: 17), AFFGMSRIGMEVTPSGTW (SEQ ID NO: 18), MEVTPSGTWL (SEQ ID NO: 19), GMSRIGMEV (SEQ ID NO: 20), ILLNKHIDA (SEQ ID NO: 21), ALNTPKDHI (SEQ ID NO: 22), IRQGTDYKHWPQIAQFA (SEQ ID NO: 23), KHWPQIAQFAPSASAFF (SEQ ID NO: 24), LALLLLDRL (SEQ ID NO: 25), LLLDRLNQL (SEQ ID NO: 26), LLNKHIDAYKTFPPTEPK (SEQ ID NO: 27), LQLPQGTTL (SEQ ID NO: 28), AQFAPSASAFFGMSR (SEQ ID NO: 29), AQFAPSASAFFGMSRIGM (SEQ ID NO: 30), RRPQGLPNNTASWFT (SEQ ID NO: 31), YKTFPPTEPKKDKKKK (SEQ ID NO: 32), GAALQIPFAMQMAYRF (SEQ ID NO: 33), MAYRFNGIGVTQNVLY (SEQ ID NO: 34), QLIRAAEIRASANLAATK (SEQ ID NO: 35), FIAGLIAIV (SEQ ID NO: 36), ALNTLVKQL (SEQ ID NO: 37), LITGRLQSL (SEQ ID NO: 38), NLNESLIDL (SEQ ID NO: 39), QALNTLVKQLSSNFGAI (SEQ ID NO: 40), RLNEVAKNL (SEQ ID NO: 41), VLNDILSRL (SEQ ID NO: 42), VVFLHVTYV (SEQ ID NO: 43)); SARS-CoV-derived spike (S) and nucleocapsid (N) protein T cell epitopes (obtained from positive MEW binding assays) that are identical in SARS-CoV-2 (including FIAGLIAIV (SEQ ID NO: 44), GLIAIVMVTI (SEQ ID NO: 45), IITTDNTFV (SEQ ID NO: 46), ALNTLVKQL (SEQ ID NO: 47), LITGRLQSL (SEQ ID NO: 48), LLLQYGSFC (SEQ ID NO: 49), LQYGSFCT (SEQ ID NO: 50), NLNESLIDL (SEQ ID NO: 51), RLDKVEAEV (SEQ ID NO: 52), RLNEVAKNL (SEQ ID NO: 53), RLQSLQTYV (SEQ ID NO: 54), VLNDILSRL (SEQ ID NO: 55), VVFLHVTYV (SEQ ID NO: 56), ILLNKHID (SEQ ID NO: 57), FPRGQGVPI (SEQ ID NO: 58), LLLLDRLNQ (SEQ ID NO: 59), GMSRIGMEV (SEQ ID NO: 60), ILLNKHIDA (SEQ ID NO: 61), ALNTPKDHI (SEQ ID NO: 62), LALLLLDRL (SEQ ID NO: 63), LLLDRLNQL (SEQ ID NO: 64), LLLLDRLNQL (SEQ ID NO: 65), LQLPQGTTL (SEQ ID NO: 66), AQFAPSASA (SEQ ID NO: 67), TTLPKGFYA (SEQ ID NO: 68), VLQLPQGTTL (SEQ ID NO: 69), GYQPYRVVVL (SEQ ID NO: 70), PYRVVVLSF (SEQ ID NO: 71), LSPRWYFYY (SEQ ID NO: 72), DSFKEELDKY (SEQ ID NO: 73), LIDLQELGKY (SEQ ID NO: 74), PYRVVVLSF (SEQ ID NO: 75), GTTLPKGFY (SEQ ID NO: 76), VTPSGTWLTY (SEQ ID NO: 77), GSFCTQLNR (SEQ ID NO: 78), GVVFLHVTY (SEQ ID NO: 79), AQALNTLVK (SEQ ID NO: 80), MTSCCSCLK (SEQ ID NO: 81), ASANLAATK (SEQ ID NO: 82), SLIDLQELGK (SEQ ID NO: 83), SVLNDILSR (SEQ ID NO: 84), TQNVLYENQK (SEQ ID NO: 85), CMTSCCSCLK (SEQ ID NO: 86), VQIDRLITGR (SEQ ID NO: 87), KTFPPTEPK (SEQ ID NO: 88), KTFPPTEPKK (SEQ ID NO: 89), LSPRWYFYY (SEQ ID NO: 90), ASAFFGMSR (SEQ ID NO: 91), ATEGALNTPK (SEQ ID NO: 92), QLPQGTTLPK (SEQ ID NO: 93), QQQGQTVTK (SEQ ID NO: 94), QQQQGQTVTK (SEQ ID NO: 95), SASAFFGMSR (SEQ ID NO: 96), SQASSRSSSR (SEQ ID NO: 97), TPSGTWLTY (SEQ ID NO: 98), FPNITNLCPF (SEQ ID NO: 99), APHGVVFLHV (SEQ ID NO: 100), FPRGQGVPI (SEQ ID NO: 101), APSASAFFGM (SEQ ID NO: 102), GAALQIPFAMQMAYR (SEQ ID NO: 103), GWTFGAGAALQIPFA (SEQ ID NO: 104), IDRLITGRLQSLQTY (SEQ ID NO: 105), ISGINASVVNIQKEI (SEQ ID NO: 106), LDKYFKNHTSPDVDL (SEQ ID NO: 107), LGDISGINASVVNIQ (SEQ ID NO: 108), LGFIAGLIAIVMVTI (SEQ ID NO: 109), LNTLVKQLSSNFGAI (SEQ ID NO: 110), LQDVVNQNAQALNTL (SEQ ID NO: 111), LQSLQTYVTQQLIRA (SEQ ID NO: 112), LQTYVTQQLIRAAEI (SEQ ID NO: 113), AQKFNGLTVLPPLLT (SEQ ID NO: 114), PCSFGGVSVITPGTN (SEQ ID NO: 115), QIPFAMQMAYRFNGI (SEQ ID NO: 116), QQLIRAAEIRASANL (SEQ ID NO: 117), QTYVTQQLIRAAEIR (SEQ ID NO: 118), AYRFNGIGVTQNVLY (SEQ ID NO: 119), SSNFGAISSVLNDIL (SEQ ID NO: 120), TGRLQSLQTYVTQQL (SEQ ID NO: 121), WLGFIAGLIAIVMVT (SEQ ID NO: 122), CVNFNFNGLTGTGVL (SEQ ID NO: 123), DKYFKNHTSPDVDLG (SEQ ID NO: 124), IDAYKTFPPTEPKKD (SEQ ID NO: 125), MSRIGMEVTPSGTWL (SEQ ID NO: 126), NKHIDAYKTFPPTEP (SEQ ID NO: 127), VLQLPQGTTLPKGFY (SEQ ID NO: 128), LQIPFAMQM (SEQ ID NO: 129), RVDFCGKGY (SEQ ID NO: 130), YEQYIKWPWY (SEQ ID NO: 131), GRLQSLQTY (SEQ ID NO: 132), VRFPNITNL (SEQ ID NO: 133)); SARS-CoV-derived linear B cell epitopes from S (23; 20 of which are located in subunit S2) and N (22) proteins that are identical in SARS-CoV-2 (including DVVNQNAQALNTLVKQL (SEQ ID NO: 134); EAEVQIDRLITGRLQSL (SEQ ID NO: 135); EIDRLNEVAKNLNESLIDLQELGKYEQY (SEQ ID NO: 136); EVAKNLNESLIDLQELG (SEQ ID NO: 137); GAALQIPFAMQMAYRFN (SEQ ID NO: 138); GAGICASY (SEQ ID NO: 139); AISSVLNDILSRLDKVE (SEQ ID NO: 140); GSFCTQLN (SEQ ID NO: 141); ILSRLDKVEAEVQIDRL (SEQ ID NO: 142); KGIYQTSN (SEQ ID NO: 143); AMQMAYRF (SEQ ID NO: 144); KNHTSPDVDLGDISGIN (SEQ ID NO: 145); MAYRFNGIGVTQNVLYE (SEQ ID NO: 146); AATKMSECVLGQSKRVD (SEQ ID NO: 147); PFAMQMAYRFNGIGVTQ (SEQ ID NO: 148); QALNTLVKQLSSNFGAI (SEQ ID NO: 149); QLIRAAEIRASANLAAT (SEQ ID NO: 150); QQFGRD (SEQ ID NO: 151); RASANLAATKMSECVLG (SEQ ID NO: 152); RLITGRLQSLQTYVTQQ (SEQ ID NO: 153); EIDRLNEVAKNLNESLIDLQELGKYEQY (SEQ ID NO: 154); SLQTYVTQQLIRAAEIR (SEQ ID NO: 155); DLGDISGINASVVNIQK (SEQ ID NO: 156)), SARS-CoV-derived discontinuous B cell epitopes (and associated known antibodies [39-41]) that have at least one site with an identical amino acid to the corresponding site in SARS-CoV-2 (including epitopes with IEDB ID 910052, 77444, and 77442); and 7D10 epitope. The delipidation process removes lipids in and around the epitopes, resulting in either a conformational change in the epitopes, epitopes adjacent to the listed epitopes to be exposed, and/or a function of the epitopes to be modified.

By substantially removing the lipid envelope of the virus, and keeping the viral particle intact, the method of the present invention exposes additional antigens. The host immune system recognizes the viral particle as foreign. Using the method of the present invention, what is created is a modified viral particle in which the antigenic core remains intact, thereby using the epitopes of the actual viral particle to initiate a positive immunogenic response in the patient into which it is reintroduced. In addition, the method of the present invention reduces the deleterious effect on the other plasma proteins, measured by protein recovery, such that the plasma can be reintroduced into the patient.

In creating this modified SARS-CoV-2 viral particle what is also created as a patient-specific antigen that induces protection against the viral particle in the species in which it is introduced. The method of the present invention creates an effective means to immunize individuals against viral pathogen infection and elicit a broad, biologically active protective immune response without risk of infecting the individual. Therefore, a vaccine may be developed from a delipidated SARS-CoV-2 virus by removing the lipid envelope and exposing antigens hidden beneath the envelope, in turn generating a positive immune response. This is referred to as an “autologous vaccine”, which can be created by the partial removal of the lipid envelope using suitable solvent systems (one which would not damage the antigens contained in the particle) exposing antigens and/or forcing a structural modification in the viral protein structures, which when introduced into the body, would provoke an effective immune response. Non-autologous vaccines may also be created in the present invention which are administered to patients that are different from the source of the virus to be delipidated. Combination vaccines directed against multiple viruses are also within the scope of the present invention. Such combination vaccines may be made from various biological fluids, from stock supplies of multiple strains or clades of a virus (e.g., different strains of SARS-CoV-2).

In one embodiment, the delipidation process is optimized to reduce lipid content in the replication-transcription complex (RTC) formed in double-membrane vesicles of SARS-CoV-2. Specifically, the solvent combination, amount of solvent used, extent of mixing time, and other processing steps disclosed herein are each selected to decrease the lipid content of the replication-transcription complex (RTC) formed in double-membrane vesicles of SARS-CoV-2 by a minimum of 5%, preferably any increment within a range 5% to 100%.

Modified, partially delipidated viral particles obtained with some embodiments of the methods disclosed herein represent, in some aspects, new therapeutic vaccine compositions for therapeutic immunization and induction of an immune response in animals or humans. In one aspect, modified, partially delipidated viral particles obtained with the methods disclosed herein are useful for therapeutic immunization and induction of an immune response in animals or humans infected by SARS-CoV-2. In one embodiment of the present invention, administration of the modified, partially delipidated viral particles and compositions comprising such particles provides a new method of treatment, alleviation, or attenuation of coronavirus infections, conditions or clinical symptoms associated with these infections such as those coronaviruses leading to the condition known as COVID-19.

Partially delipidated SARS-CoV-2 viral particles obtained according to some of aspects of the present invention possess at least some structural characteristics that distinguish them from the conventional delipidated viruses. Such characteristics include, but are not limited to, the content of viral proteins, including viral envelope proteins or host viral membrane associated proteins, the cholesterol content of the partially delipidated viral particles, or the ratio of cholesterol content to viral protein. For example, a partially delipidated coronavirus viral particle according to some embodiments of the present invention has a lower cholesterol content than the cholesterol content of the non-delipidated coronavirus viral particle. In one embodiment, the lower cholesterol content of the partially delipidated SARS-CoV-2 coronavirus viral particle can be at least 20% to 30% lower than the cholesterol content of the non-delipidated coronavirus viral particle. In other embodiments, the cholesterol content in the modified, partially delipidated coronavirus viral particle is reduced, for example, no more than 80%, 60%, 55%, or 50% as compared to the unmodified viral particle. In other embodiments, the protein content in the modified, partially delipidated coronavirus viral particle is reduced, for example, no more than 5%, 10%, 15%, 20%, 30%, 40%, 50% or 55% as compared to the unmodified coronavirus viral particle. According to other embodiments, the modified, partially delipidated coronavirus viral particle has a ratio of cholesterol total protein of at least 0.06.

One of ordinary skill in the art would appreciate that there may be multiple delipidation processes employed under the scope of this invention. In a preferred embodiment, a solvent system together with applied energy, for example a mechanical mixing system, is used to substantially delipidate the viral particle. The delipidation process is dependent upon the total amount of solvent and energy input into a system. Various solvent levels and mixing methods, as described below, may be used depending upon the overall framework of the process.

The solvent or combinations of solvents to be employed in the process of partially or completely delipidating SARS-CoV-2 viruses may be any solvent or combination of solvents effective in solubilizing lipids in the viral envelope while retaining the structural integrity of the modified SARS-CoV-2 viral particle, which can be measured, in one embodiment, via protein recovery. A delipidation process falling within the scope of the present invention uses an optimal combination of energy input and solvent to delipidate the SARS-CoV-2 viral particle, while still keeping it intact. Suitable solvents comprise hydrocarbons, ethers, fluoroethers, alcohols, phenols, esters, halohydrocarbons, halocarbons, amines, detergents, surfactants, and mixtures thereof. Aromatic, aliphatic, or alicyclic hydrocarbons may also be used. Other suitable solvents, which may be used with the present invention, include amines and mixtures of amines. One solvent system is DIPE, either concentrated or diluted in water or a buffer such as a physiologically acceptable buffer. One solvent combination comprises alcohols and ethers. Another solvent comprises ether or combinations of ethers and a surfactant, such as polyethylene glycol p-(1,1,3,3-tetramethylbutyl)-phenyl ether (also known and referred to herein as TRITON X-100™ or polyoxyethylene octyl phenyl ether). Another solvent comprises ether or combinations of ethers, either in the form of symmetrical ethers, asymmetrical ethers or halogenated ethers such as fluoroethers.

Suitable first extraction solvents include solvents that extract or dissolve lipid, including but not limited to alcohols, hydrocarbons, amines, ethers, fluoroethers (including but not limited to fluoromethyl hexafluoroisopropyl ether (sevoflurane)), surfactants, detergents, and combinations thereof. First extraction solvents may be combinations such as the following: 1) an alcohol and an ether; 2) an alcohol and a fluoroether; 3) an alcohol and a surfactant, 4) an ether and a surfactant; or 5) an alcohol, an ether and a surfactant. First extraction solvents include, but are not limited to n-butanol, di-isopropyl ether (DIPE), fluoroether such as sevoflurane, surfactants such as TRITON X-100™ (polyethylene glycol p-(1,1,3,3-tetramethylbutyl)-phenyl ether or polyoxyethylene octyl phenyl ether) or Tween 20 (PEG(20) sorbitan monolaurate), diethyl ether, and combinations thereof.

The optimal solvent systems are those that accomplish two objectives: first, at least partially delipidating SARS-CoV-2 virus and second, employing a set of conditions such that there are few or no deleterious effects on the other plasma proteins. In addition, the solvent system should maintain the integrity of the SARS-CoV-2 viral particle such that it can be used to initiate an immune response in the patient. It should therefore be noted that certain solvents, solvent combinations, and solvent concentrations may be too harsh to use in the present invention because they result in a chemical kill.

It is preferred that the solvent or combination of solvents has a relatively low boiling point to facilitate removal through a vacuum and possibly heat without destroying the antigenic core of the viral particle. It is also preferred that the solvent or combination of solvents be employed at a low temperature because heat has deleterious effects on the proteins contained in biological fluids such as plasma. It is also preferred that the solvent or combination of solvents at least partially delipidate the SARS-CoV-2 viral particle.

Liquid hydrocarbons dissolve compounds of low polarity such as the lipids found in the viral envelopes of the infectious organisms. Particularly effective in disrupting the lipid membrane of a viral particle are hydrocarbons which are substantially water immiscible and liquid at about 37 C. Suitable hydrocarbons include, but are not limited to the following: C₅ to C₂₀ aliphatic hydrocarbons such as petroleum ether, hexane, heptane, octane; haloaliphatic hydrocarbons such as chloroform, 1,1,2-trichloro-1,2,2-trifluoroethane, 1,1,1-trichloroethane, trichloroethylene, tetrachloroethylene, dichloromethane and carbon tetrachloride; thioaliphatic hydrocarbons each of which may be linear, branched or cyclic, saturated or unsaturated; aromatic hydrocarbons such as benzene; ketones; alkylarenes such as toluene; haloarenes; haloalkylarenes; and thioarenes. Other suitable solvents may also include saturated or unsaturated heterocyclic compounds such as pyridine and aliphatic, thio- or halo-derivatives thereof.

Suitable esters for use in the present invention include, but are not limited to, ethyl acetate, propylacetate, butylacetate and ethylpropionate. Suitable detergents/surfactants that may be used include but are not limited to the following: sulfates, sulfonates, phosphates (including phospholipids), carboxylates, and sulfosuccinates. Some anionic amphiphilic materials useful with the present invention include but are not limited to the following: sodium dodecyl sulfate (SDS), sodium decyl sulfate, bis-(2-ethylhexyl) sodium sulfosuccinate (AOT), cholesterol sulfate and sodium laurate.

Solvents may be removed from delipidated SARS-CoV-2 viral mixtures through the use of additional solvents. For example, demulsifying agents such as ethers may be used to remove a first solvent such as an alcohol from an emulsion. Removal of solvents may also be accomplished through other methods, which do not employ additional solvents, including but not limited to the use of charcoal. Charcoal may be used in a slurry or alternatively, in a column to which a mixture is applied. Charcoal is a preferred method of removing solvents. Pervaporation may also be employed to remove one or more solvents from delipidated viral mixtures.

Examples of suitable amines for use in removal of lipid from lipid-containing organisms in the present invention are those which are substantially immiscible in water. Typical amines are aliphatic amines—those having a carbon chain of at least 6 carbon atoms. A non-limiting example of such an amine is C₆H₁₃N₂.

Ether is a preferred solvent for use in the method of the present invention. Particularly preferred are the C₄-C₈ containing-ethers, including but not limited to ethyl ether, diethyl ether, and propyl ethers (including but not limited to di-isopropyl ether (DIPE)). Asymmetrical ethers may also be employed. Halogenated symmetrical and asymmetrical ethers may also be employed. Fluoroethers including but not limited to fluoromethyl hexafluoroisopropyl ether may also be employed. Halogenated symmetrical and asymmetrical ethers, such as fluoroethers may be employed alone or in combination with other solvents in different ratios such as sevoflurane:DIPE ratios of 0.01 parts sevoflurane to 99.99 parts DIPE to 60 parts sevoflurane to 40 parts DIPE, with a specific ratio range of about 10 parts sevoflurane to 90 parts DIPE to 5 parts sevoflurane to 95 parts DIPE, with a specific ratio range of about 10 parts sevoflurane to 90 parts DIPE to 50 parts sevoflurane to 50 parts DIPE, with a specific ratio range of about 20 parts sevoflurane to 80 parts DIPE to 45 parts sevoflurane to 55 parts DIPE, with a specific range of about 25 parts sevoflurane to 75 parts DIPE.

Other ratios include sevoflurane:n-butanol ratios of 0.01 parts sevoflurane to 99.99 parts n-butanol to 60 parts sevoflurane to 40 parts n-butanol, with a specific ratio range of about 10 parts sevoflurane to 90 parts n-butanol to 5 parts sevoflurane to 95 parts n-butanol, with a specific ratio range of about 10 parts sevoflurane to 90 parts n-butanol to 50 parts sevoflurane to 50 parts n-butanol, with a specific ratio range of about 20 parts sevoflurane to 80 parts n-butanol to 45 parts sevoflurane to 55 parts n-butanol, with a specific range of about 25 parts sevoflurane to 75 parts n-butanol.

Low concentrations of solvents, such as ethers, may be employed to remove lipids when used alone and not in combination with other solvents. For example, a low concentration range of solvents, such as ethers includes but is not limited to 0.5% to 30%, 0.01% to 10%, 0.01% to 5%, 0.1% to 5%, 0.01% to 2%, or 0.1% to 2%, or any number within these ranges. Specific concentrations of solvents, such as ethers, that may be employed include, but are not limited to the following: 0.1%, 0.625%, 1.0% 1.25%, 2%, 2.5%, 3.0%, 3.5%, 5.0% and 10% or higher. It has been observed that dilute solutions of solvents, such as ethers, are effective. Such solutions may be aqueous solutions or solutions in aqueous buffers, such as phosphate buffered saline (PBS). Other physiological buffers may be used, including but not limited to bicarbonate, citrate, Tris, Tris/EDTA, and Trizma. Preferred ethers are di-isopropyl ether (DIPE) and diethyl ether (DEE). Low concentrations of ethers may also be used in combination with alcohols, for example, n-butanol.

When used in the present invention, appropriate alcohols are those which are not appreciably miscible with plasma or other biological fluids. Such alcohols include, but are not limited to, straight chain and branched chain alcohols, including pentanols, hexanols, heptanols, octanols and those alcohols containing higher numbers of carbons.

When alcohols are used in combination with another solvent, for example, an ether, a hydrocarbon, an amine, or a combination thereof, C₁-C₈ containing alcohols may be used. Alcohols for use in combination with another solvent include C₄-C₈ containing alcohols. Accordingly, alcohols that fall within the scope of the present invention are butanols, pentanols, hexanols, heptanols and octanols, and iso forms thereof, in particular, C₄ alcohols or butanols (1-butanol and 2-butanol). The specific alcohol choice is dependent on the second solvent employed.

Ethers and alcohols can be used in combination as a first solvent for treating the fluid containing the lipid-containing virus, or viral particle. Any combination of alcohol and ether may be used provided the combination is effective to at least partially remove lipid from the infectious organism, without having deleterious effects on the plasma proteins. In one embodiment, lipid is removed from the viral envelope of the infectious organism. When alcohols and ether are combined as a first solvent for treating the infectious organism contained in a fluid, ratios of alcohol to ether in this solvent range from about 0.01 parts alcohol to 99.99 parts ether to 60 parts alcohol to 40 parts ether, with a specific ratio range of about 10 parts alcohol to 90 parts ether to 5 parts alcohol to 95 parts ether, with a specific ratio range of about 10 parts alcohol to 90 parts ether to 50 parts alcohol to 50 parts ether, with a specific ratio range of about 20 parts alcohol to 80 parts ether to 45 parts alcohol to 55 parts ether, with a specific range of about 25 parts alcohol to 75 parts ether.

One combination of alcohol and ether is the combination of butanol and di-isopropyl ether (DIPE). When butanol and DIPE are combined as a first solvent for treating the infectious organism contained in a fluid, ratios of butanol to DIPE in this solvent are about 0.01 parts butanol to 99.99 parts DIPE to 60 parts butanol to 40 parts DIPE, with a specific ratio range of about 10 parts butanol to 90 parts DIPE to 5 parts butanol to 95 parts DIPE, with a specific ratio range of about 10 parts butanol to 90 parts DIPE to 50 parts butanol to 50 parts DIPE, with a specific ratio range of about 20 parts butanol to 80 parts DIPE to 45 parts butanol to 55 parts DIPE, with a specific range of about 25 parts butanol to 75 parts DIPE.

Another combination of alcohol and ether is the combination of butanol with diethyl ether (DEE). When butanol is used in combination with DEE as a first solvent, ratios of butanol to DEE are about 0.01 parts butanol to 99.99 parts DEE to 60 parts butanol to 40 parts DEE, with a specific ratio range of about 10 parts butanol to 90 parts DEE to 5 parts butanol to 95 parts DEE with a specific ratio range of about 10 parts butanol to 90 parts DEE to 50 parts butanol to 50 parts DEE, with a specific ratio range of about 20 parts butanol to 80 parts DEE to 45 parts butanol to 55 parts DEE, with a specific range of about 40 parts butanol to 60 parts DEE. This combination of about 40% butanol and about 60% DEE (vol:vol) has been shown to have no significant effect on a variety of biochemical and hematological blood parameters, as shown for example in U.S. Pat. No. 4,895,558.

Surfactants such anionic and nonionic surfactants may also be employed alone or together with other solvents. Nonionic surfactants are known to one of ordinary skill in the art and may include without limitation surfactants known as Triton, for example TRITON X-100™ (polyethylene glycol p-(1,1,3,3-tetramethylbutyl)-phenyl ether or polyoxyethylene octyl phenyl ether), Tweens such as Tween 20 (PEG(20) sorbitan monolaurate), or Pluronic (block copolymers based on ethylene oxide and propylene oxide). When employed alone, or together with other solvents such as ethers or lower order alcohols, for example DIPE or n-butanol or combinations thereof, surfactants may be used in concentrations of from 0.001% to 1%, 0.07% to 0.8%, 0.05% to 0.5%, or 0.03% to 0.3%.

As stated above, various biological fluids may be treated with the method of the present invention in order to reduce the levels of infectivity of the lipid-containing organism in the biological fluid and to create modified SAR-CoV-2 viral particles. In a preferred embodiment, plasma obtained from an animal or human is treated with the method of the present invention in order to reduce the concentration and/or infectivity of SAR-CoV-2 viruses within the plasma and to create modified SAR-CoV-2 viral particles. In this embodiment, plasma may be obtained from an animal or human patient by withdrawing blood from the patient using well-known methods and treating the blood in order to separate the cellular components of the blood (red and white cells) from the plasma. Such methods for treating the blood are known to one of ordinary skill in the art and include but are not limited to centrifugation and filtration. Use of the present invention permits treatment of SAR-CoV-2 viral particles, for example those found within plasma, without having deleterious effects on other plasma proteins and maintaining the integrity of the viral core.

SAR-CoV-2 viruses in the plasma are affected by the treatment of the plasma with the method of the present invention. The lipid-containing viral organism may be separated from the red and white cells using techniques known to one of ordinary skill in the art.

Biological fluids include stocks of viral preparations including various strains of SAR-CoV-2 viruses. Treatment of such biological fluids with the method of the present invention produces modified viral particles that may be administered to a patient as a non-autologous vaccine. Such non-autologous vaccines provide protection in the patient against more than strain of a virus and/or against more than one type of virus. Treatment of lipid-containing organisms may occur in biological fluids other than blood and plasma. For example, peritoneal fluid may be treated with the present invention to affect the levels and infectivity of lipid-containing organisms without deleterious effects on protein components. The treated fluid may subsequently be reintroduced into the animal or human from which it was obtained. Treatment of non-blood types of fluids affects the lipid-containing organisms in the fluid, such as the virus.

Once a biological fluid, such as plasma, is obtained either in this manner, or for example, from a storage facility housing bags of plasma, the plasma is contacted with a first organic solvent, as described above, capable of solubilizing lipid in the lipid-containing infectious organism. The first organic solvent is combined with the plasma in a ratio wherein the first solvent is present in an amount effective to substantially solubilize the lipid in the infectious organism, for example, dissolve the lipid envelope that surrounds the virus. Exemplary ratios of first solvent to plasma (expressed as a ratio of first organic solvent to plasma) are described in the following ranges: 0.5-4.0:0.5-4.0; 0.8-3.0:0.8-3.0; and 1-2:0.8-1.5. Various other ratios may be applied, depending on the nature of the biological fluid. For example, in the case of cell culture fluid, the following ranges may be employed of first organic solvent to cell culture fluid: 0.5-4.0:0.5-4.0; 0.8-3.0:0.8-3.0; and 1-2:0.8-1.5.

After contacting the fluid containing the infectious organism with the first solvent as described above, the first solvent and fluid are mixed, using methods including but not limited to one of the following suitable mixing methods: gentle stirring; vigorous stirring; vortexing; swirling; homogenization; and, end-over-end rotation.

The amount of time required for adequate mixing of the first solvent with the fluid is related to the mixing method employed. Fluids are mixed for a period of time sufficient to permit intimate contact between the organic and aqueous phases, and for the first solvent to at least partially or completely solubilize the lipid contained in the SAR-CoV-2 viruses. Typically, mixing will occur for a period of about 10 seconds to about 24 hours, possibly about 10 seconds to about 2 hours, possibly approximately 10 seconds to approximately 10 or 20 minutes, or possibly about 30 seconds to about 1 hour, depending on the mixing method employed. Non-limiting examples of mixing durations associated with different methods include 1) gentle stirring and end-over-end rotation for a period of about 10 seconds to about 24 hours, 2) vigorous stirring and vortexing for a period of about 10 seconds to about 30 minutes, 3) swirling for a period of about 10 seconds to about 2 hours, or 4) homogenization for a period of about 10 seconds to about 10 minutes.

After mixing of the first solvent with the fluid, the solvent is separated from the fluid being treated. The organic and aqueous phases may be separated by any suitable manner known to one of ordinary skill in the art. Since the first solvent is typically immiscible in the aqueous fluid, the two layers are permitted to separate and the undesired layer is removed. The undesired layer is the solvent layer containing dissolved lipids and its identification, as known to one of ordinary skill in the art, depends on whether the solvent is more or less dense than the aqueous phase. An advantage of separation in this manner is that dissolved lipids in the solvent layer may be removed.

In addition, separation may be achieved through means, including but not limited to the following: removing the undesired layer via pipetting; centrifugation followed by removal of the layer to be separated; creating a path or hole in the bottom of the tube containing the layers and permitting the lower layer to pass through; utilization of a container with valves or ports located at specific lengths along the long axis of the container to facilitate access to and removal of specific layers; and any other means known to one of ordinary skill in the art. Another method of separating the layers, especially when the solvent layer is volatile, is through distillation under reduced pressure or evaporation at room temperature, optionally combined with mild heating. In one embodiment employing centrifugation, relatively low g forces are employed, such as 900 g for about 5 to 15 minutes to separate the phases.

A preferred method of removing solvent is through the use of charcoal, preferably activated charcoal. This charcoal is optionally contained in a column. Alternatively the charcoal may be used in slurry form. Various biocompatible forms of charcoal may be used in these columns. Pervaporation methods and use of charcoal to remove solvents are preferred methods for removing solvent.

Following separation of the first solvent from the treated fluid, some of the first solvent may remain entrapped in the aqueous layer as an emulsion. A preferred method of removing a first solvent or a demulsifying agent is through the use of adsorbents, such as charcoal. The charcoal is preferably activated charcoal. This charcoal is optionally contained in a column, as described above. Still another method of removing solvent is the use of hollow fiber contactors. Pervaporation methods and charcoal adsorbant methods of removing solvents are preferred. In yet another embodiment, a de-emulsifying agent is employed to facilitate removal of the trapped first solvent. The de-emulsifying agent may be any agent effective to facilitate removal of the first solvent. A preferred de-emulsifying agent is ether and a more preferred de-emulsifying agent is diethyl ether. The de-emulsifying agent may be added to the fluid or in the alternative the fluid may be dispersed in the de-emulsifying agent. In vaccine preparation, alkanes in a ratio of about 0.5 to 4.0 to about 1 part of emulsion (vol:vol) may be employed as a de-emulsifying agent, followed by washing to remove the residual alkane from the remaining delipidated organism used for preparing the vaccine. Preferred alkanes include, but are not limited to, pentane, hexane and higher order straight and branched chain alkanes.

The de-emulsifying agent, such as ether, may be removed through means known to one of skill in the art, including such means as described in the previous paragraph. One convenient method to remove the de-emulsifying agent, such as ether, from the system, is to permit the ether to evaporate from the system in a running fume hood or other suitable device for collecting and removing the de-emulsifying agent from the environment. In addition, de-emulsifying agents may be removed through application of higher temperatures, for example from about 24 to 37 C. with or without pressures of about 10 to 20 mbar. Another method to remove the de-emulsifying agent involves separation by centrifugation, followed by removal of organic solvent through aspiration, further followed by evaporation under reduced pressure (for example 50 mbar) or further supply of an inert gas, such as nitrogen, over the meniscus to aid in evaporation.

It is to be understood that the method of the present invention may be employed in either a continuous or discontinuous manner. That is, in a continuous manner, a fluid may be fed to a system employing a first solvent which is then mixed with the fluid, separated, and optionally further removed through application of a de-emulsifying agent. The continuous method also facilitates subsequent return of the fluid containing delipidated infectious organism to a desired location. Such locations may be containers for receipt and/or storage of such treated fluid, and may also include the vascular system of a human or animal or some other body compartment of a human or animal, such as the pleural, pericardial, peritoneal, and abdominopelvic spaces.

In one embodiment of the continuous method of the present invention, a biological fluid, for example, blood, is removed from an animal or a human through means known to one of ordinary skill in the art, such as a catheter. Appropriate anti-clotting factors as known to one of ordinary skill in the art are employed, such as heparin, ethylenediaminetetraacetic acid (EDTA) or citrate. This blood is then separated into its cellular and plasma components through the use of a centrifuge. The plasma is then contacted with the first solvent and mixed with the first solvent to effectuate lipid removal from the infectious organism contained within the plasma. Following separation of the first solvent from the treated plasma, charcoal, pervaporation or a de-emulsifying agent is optionally employed to remove entrapped first solvent. After ensuring that acceptable levels (non-toxic) of first solvent or de-emulsifying agent, if employed, are found within the plasma containing the delipidated infectious organism, the plasma is then optionally combined with the cells previously separated from the blood to form a new blood sample containing at least partially delipidated viral particles, also called modified viral particles herein.

Through the practice of this method, the infectivity of the infectious organism is greatly reduced or eliminated. Following recombination with the cells originally separated from the blood, the fluid with reduced lipid levels and containing virus with reduced lipid levels may be reintroduced into either the vascular system or some other system of the human or animal. The effect of such treatment of plasma removed from the human or animal and return of the sample containing the partially or completely delipidated infectious organism, or modified viral particle, to the human or animal causes a net decrease in the infectivity of the infectious organism contained within the vascular system of the human or animal. The modified viral particle also serves to initiate an autologous immune response in the patient when administered to the patient. In this mode of operation, the method of the present invention is employed to treat body fluids in a continuous manner—while the human or animal is connected to an extracorporeal device for such treatment.

In yet another embodiment, the discontinuous or batch mode, the human or animal is not connected to an extracorporeal device for processing bodily fluids with the method of the present invention. In a discontinuous mode of operation, the present invention employs a fluid previously obtained from a human or animal, which may include, but is not limited to plasma, lymphatic fluid, or follicular fluid. The fluid may be contained within a blood bank or in the alternative, drawn from a human or animal prior to application of the method. The fluid may be a reproductive fluid or any fluid used in the process of artificial insemination or in vitro fertilization. The fluid may also be one not directly obtained from a human or animal but rather any fluid containing a potentially infectious organism, such as cell culture fluid. Stocks of various strains or clades of a virus and also stocks of multiple viruses may be used in the present method to produce vaccines. In this mode of operation, this fluid is treated with the method of the present invention to produce a new fluid with reduced lipid levels which contains at least partially or completely delipidated SAR-CoV-2 viral particles. One embodiment of this mode of the present invention is to treat plasma samples previously obtained from other animals or humans and stored in a blood bank for subsequent transfusion. This is a non-autologous method of providing vaccine protection. These samples may be treated with the method of the present invention to treat or prevent one or more infectious disease, such as SARS, HIV, hepatitis, and/or cytomegalovirus, from the biological sample.

Delipidation of an infectious organism can be achieved by various means. A batch method can be used for fresh or stored biological fluids, for example, fresh frozen plasma. In this case a variety of the described organic solvents or mixtures thereof can be used for viral inactivation. Extraction time depends on the solvent or mixture thereof and the mixing procedure employed.

Through the use of the methods of the present invention, levels of lipid in SAR-CoV-2 viruses in a fluid are reduced, and the fluid, for example, delipidated plasma containing the modified SAR-CoV-2 viral particles may be administered to the patient. Such fluid contains modified SAR-CoV-2 viral particles with reduced infectivity, act as a vaccine and provide protection in the patient against the virus or provide a treatment in an infected patient by generating an immune response and decreasing the severity of the disease. These modified SAR-CoV-2 viral particles induce an immune response in the recipient to exposed epitopes on the modified viral particles. Alternatively, the modified SAR-CoV-2 viral particles may be combined with a pharmaceutically acceptable carrier, and optionally an adjuvant, and administered as a vaccine composition to a human or an animal to induce an immune response in the recipient.

In one embodiment, the modified SAR-CoV-2 particle, which is at least partially or substantially delipidated and has immunogenic properties, is optionally combined with a pharmaceutically acceptable carrier to make a composition comprising a vaccine. In a preferred embodiment, the modified SAR-CoV-2 viral particle is retained in the biological fluid, such as plasma, with reduced lipid levels and is administered to a patient as a vaccine. This vaccine composition is optionally combined with an adjuvant or an immunostimulant and administered to an animal or a human. Both autologous and non-autologous vaccines, including combination vaccines, are within the scope of the present invention. It is to be understood that vaccine compositions may contain more than one type of modified SAR-CoV-2 viral particle or component thereof, in order to provide protection against more than one strain of a virus or more than one viral disease after vaccination. Such combinations may be selected according to the desired immunity. More specifically, the vaccine can comprise a plurality of SAR-CoV-2 viral particles having patient-specific antigens and modified SAR-CoV-2 viral particles having non-patient specific antigens or stock viral particles that have undergone the delipidation process of the present invention. The remaining modified viral particles of the organism are retained in the delipidated biological fluid, and when reintroduced into the animal or human, are presumably ingested by phagocytes and generate an immune response.

When a delipidated infectious organism, for example one in the form of a modified SAR-CoV-2 viral particle with exposed antigenic determinants, is administered to an animal or a human, it is optionally combined with a pharmaceutically acceptable carrier to produce a vaccine, and optionally combined with an adjuvant or an immunostimulant as known to one of ordinary skill in the art. The vaccine formulations may conveniently be presented in unit dosage form and may be prepared by conventional pharmaceutical techniques known to one of ordinary skill in the art. Such techniques include uniformly and intimately bringing into association the active ingredient and the liquid carriers (pharmaceutical carrier(s) or excipient(s)). Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers—for example, sealed ampules and vials—and may be stored in a freeze-dried (lyophilized) condition requiring only the addition of the sterile liquid carrier, for example, water for injections, immediately prior to use. The vaccine may be stored at temperatures of from about 4° C. to −100° C. The vaccine may also be stored in a lyophilized state at different temperatures including room temperature. Extemporaneous injection solutions and suspensions may be prepared from sterile powders, granules and tablets commonly used by one of ordinary skill in the art. The vaccine may be sterilized through conventional means known to one of ordinary skill in the art. Such means include, but are not limited to filtration, radiation and heat. The vaccine of the present invention may also be combined with bacteriostatic agents, such as thimerosal, to inhibit bacterial growth.

Preferred unit dosage formulations are those containing a dose or unit, or an appropriate fraction thereof, of the administered ingredient. It should be understood that in addition to the ingredients, particularly mentioned above, the formulations of the present invention may include other agents commonly used by one of ordinary skill in the art.

The vaccine may be administered through different routes, such as oral, including buccal and sublingual, rectal, parenteral, aerosol, nasal, intramuscular, subcutaneous, intradermal, intravenous, intraperitoneal, and topical. The vaccine may also be administered in the vicinity of lymphatic tissue, for example through administration to the lymph nodes such as axillary, inguinal or cervical lymph nodes.

The vaccine of the present invention may be administered in different forms, including but not limited to solutions, emulsions and suspensions, microspheres, particles, microparticles, nanoparticles, and liposomes. It is expected that from about 1 to 5 dosages may be required per immunization regimen. One of ordinary skill in the medical or veterinary arts of administering vaccines will be familiar with the amount of vaccine to be administered in an initial injection and in booster injections, if required, taking into consideration, for example, the age and size of a patient. Initial injections may range from about less than 1 ng to 1 gram based on total viral protein. A non-limiting range may be 1 ml to 10 ml. The volume of administration may vary depending on the administration route.

The vaccines of the present invention may be administered before, during or after an infection. The vaccine of the present invention may be administered to either humans or animals. In one embodiment, the viral load (one or more viruses) of a human or an animal may be reduced by delipidation treatment of the plasma. The same individual may receive a vaccine directed to the one or more viruses, thereby stimulating the immune system to combat against the virus that remains in the individual. The time for administration of the vaccine before initial infection is known to one of ordinary skill in the art. However, the vaccine may also be administered after initial infection to ameliorate disease progression or to treat the disease.

A variety of adjuvants known to one of ordinary skill in the art may be administered in conjunction with the modified viral particles in the vaccine composition. Such adjuvants include, but are not limited to the following: polymers, co-polymers such as polyoxyethylene-polyoxypropylene co-polymers, including block co-polymers; polymer P1005; monotide ISA72; Freund's complete adjuvant (for animals); Freund's incomplete adjuvant; sorbitan monooleate; squalene; CRL-8300 adjuvant; alum; QS 21, muramyl dipeptide; trehalose; bacterial extracts, including mycobacterial extracts; detoxified endotoxins; membrane lipids; water-in-oil mixtures, water-in-oil-in-water mixtures or combinations thereof.

A variety of suspending fluids or carriers known to one of ordinary skill in the art may be employed to suspend the vaccine composition. Such fluids include without limitation: sterile water, saline, buffer, or complex fluids derived from growth medium or other biological fluids. Preservatives, stabilizers and antibiotics known to one of ordinary skill in the art may be employed in the vaccine composition.

The following experimental examples are illustrative in showing that a delipidation process of the viral particle occurred and in particular, that the viral particle was modified and noted to exhibit a positive immunogenic response in the species from which it was derived. It will be appreciated that other embodiments and uses will be apparent to those skilled in the art and that the invention is not limited to these specific illustrative examples or preferred embodiments.

### System for Delipidating SARS-CoV-2

Embodiments of the present specification provide systems and methods to achieve the above objectives. Systems and methods are provided where plasma and solvent(s) are introduced into a specially designed mixing bag in precise quantities and volumetric ratios. The solvent and plasma are then mixed in an orbital fashion for a prescribed period, resulting in delipidation. The mixture is then drained into a separator bag. Each batch is mixed and drained into the separator bag until the input plasma is fully processed. When the separator bag reaches capacity, excess solvent is drained to a solvent waste bag.

The timed suspension in the separator bag separates the plasma and solvent into distinct fractions so the solvent can be drained into the solvent waste bag. Some solvent, however, remains dissolved in the plasma. This residual solvent is substantially removed by passing the plasma through a specially-designed charcoal column. The output plasma contains delipidated SARS-CoV-2.

FIG. 1 illustrates an exemplary prior art system and its components used to achieve the methods of the present specification. The figure depicts an exemplary basic component flow diagram defining elements of the SARS-CoV-2 modification system 100. Embodiments of the components of system 100 are utilized after obtaining a blood fraction from a patient or another individual (donor). The plasma separated from the blood is brought in a sterile bag to system 100 for further processing. The plasma may be separated from blood using a known plasmapheresis device. The plasma may be collected from the patient into a sterile bag using standard apheresis techniques. The plasma is then brought in the form of a fluid input to system 100 for further processing. In embodiments, system 100 is not connected to the patient at any time and is a discrete, stand-along system for delipidating SARS-CoV-2 viral particles in plasma. The patient's plasma is processed by system 100 and brought back to the patient's location to be reinfused back into the patient. In alternate embodiments, the system may be a continuous flow system that is connected to the patient in which both plasmapheresis and delipidation are performed in an excorporeal, parallel system and the delipidated plasma product is returned to the patient.

A fluid input 105 (containing blood plasma) is provided and connected via tubing to a mixing device 120. A solvent input 110 is provided and also connected via tubing to mixing device 120. In embodiments, valves 115, 116 are used to control the flow of fluid from fluid input 105 and solvent from solvent input 110 respectively. It should be appreciated that the fluid input 105 contains any fluid that includes SARS-CoV-2 viral particles, including plasma. It should further be appreciated that solvent input 110 can include a single solvent, a mixture of solvents, or a plurality of different solvents that are mixed at the point of solvent input 110. While depicted as a single solvent container, solvent input 110 can comprise a plurality of separate solvent containers. Embodiments of types of solvents that may be used are discussed subsequently.

Mixer 120 mixes fluid from fluid input 105 and solvent from solvent input 110 to yield a fluid-solvent mixture. In some embodiments, mixer 120 is capable of using a shaker bag mixing method with the input fluid and input solvent in a plurality of batches, such as 1, 2, 3 or more batches. In alternative embodiments, other known methods of mixing are utilized. Once formed, the fluid-solvent mixture is directed, through tubing and controlled by at least one valve 115a, to a separator 125. In an embodiment, separator 125 is capable of performing bulk solvent separation through gravity separation in a funnel-shaped bag.

In separator 125, the fluid-solvent mixture separates into a first layer and second layer. The first layer comprises a mixture of solvent and lipid that has been removed from the SARS-CoV-2 viral particles. Typically, the solvent is heavier than the plasma and therefore the solvent settles at the bottom of separator 125, and the delipidated SARS-CoV-2 viral particles in the plasma is at the top. In embodiments, the density/specific gravity of solvent is approximately 1.5 times greater than that of the plasma fluid. In embodiments, separator 125 is conical or V-shaped. Once the solvent settles at the bottom, it can be drained from separator 125 while the plasma fluid containing SARS-CoV-2 viral particles is retained. The first layer is transported through a valve 115b to a first waste container 135. The second layer comprises a mixture of residual solvent, modified SARS-CoV-2 viral particles, and other elements of the input fluid. One of ordinary skill in the art would appreciate that the composition of the first layer and the second layer would differ based upon the nature of the input fluid. Once the first and second layers separate in separator 125, the second layer is transported through tubing to a solvent extraction device 140. In an embodiment, a pressure sensor (not shown) and valve 130 is positioned in the flow stream to control the flow of the second layer to solvent extraction device 140.

The opening and closing of valves 115, 116 to enable the flow of fluid from input containers 105, 110 may be timed using mass balance calculations derived from weight determinations of the fluid inputs 105, 110, and separator 125. For example, valve 115b between separator 125 and first waste container 135 and valve 130 between separator 125 and solvent extraction device 140 open after the input masses (fluid and solvent) substantially balances with the mass in separator 125 and a sufficient period of time has elapsed to permit separation between the first and second layers. Depending on what solvent is used, and therefore which layer settles to the bottom of separator 125, either valve 115b between separator 125 and first waste container 135 is opened or valve 130 between separator 125 and solvent extraction device 140 is opened. One of ordinary skill in the art would appreciate that the timing of the opening is dependent upon how much fluid is in the first and second layers and would further appreciate that it is preferred to keep valve 115b between separator 125 and first waste container 135 open just long enough to remove all of the first layer and some of the second layer, thereby ensuring that as much solvent as possible has been removed from the fluid being sent to solvent extraction device 140.

In embodiments, an infusion grade fluid (“IGF”) may be employed via one or more inputs 160 which are in fluid communication with the fluid path 121 leading from separator 125 to solvent extraction device 140 for priming. In an embodiment, saline is employed as the infusion grade priming fluid in at least one of inputs 160. In an embodiment, 0.9% sodium chloride (saline) is employed. In other embodiments, glucose may be employed as the infusion grade priming fluid in any one of inputs 160.

A plurality of valves 115c and 115d are also incorporated in the flow stream from glucose input 155 and saline input 160 respectively, to the tubing providing the flow path 121 from separator 125 to solvent extraction device 140. Infusion grade fluid such as saline and/or glucose is incorporated into embodiments of the present specification in order to prime solvent extraction device 140 prior to operation of the system. In embodiments, saline is used to prime most of the fluid communication lines and solvent extraction device 140. If priming is not required, the infusion grade fluid inputs are not employed. Where such priming is not required, the glucose and saline inputs are not required. In an embodiment, priming is not required in the lines between a second waste container 165 and output container 145. Also, one of ordinary skill in the art would appreciate that the glucose and saline inputs can be replaced with other primers if required by the solvent extraction device 140.

In some embodiments, solvent extraction device 140 is a charcoal column designed to remove the specific solvent used in solvent input 110. Exemplary solvent extraction device 140 includes but is not limited to an Asahi Hemosorber™ charcoal column or the Baxter/Gambro Adsorba™ 300C charcoal column or any other charcoal column that is employed in blood hemoglobin perfusion procedures. In embodiments, it should be noted that if the charcoal column is pre-primed with glucose, it will limit the amount of glucose removed from plasma because the free glucose in the priming agent will bind to glucose sites in the charcoal column, limiting its ability to absorb more glucose. A pump 150 is used to move the second layer from separator 125, through solvent extraction device 140, and to output container 145, through a U-shaped configuration. In embodiments, pump 150 is a rotary peristaltic pump, such as a Masterflex Model 77201-62.

The first layer is directed to waste container 135 that is in fluid communication with separator 125 through tubing and at least one valve 115b. Additionally, other waste, if generated, can be directed from the fluid path connecting solvent extraction device 140 and output container 145 to second waste container 165. Optionally, in an embodiment, a valve 115f is included in the path from the solvent extraction device 140 to the output container 145. Optionally, in an embodiment, a valve 115g is included in the path from the solvent extraction device 140 to the second waste container 165.

In an embodiment of the present specification, gravity is used, wherever practical, to move fluid through each of the plurality of components. For example, gravity is used to drain input plasma 105 and input solvent 110 into mixer 120. Where mixer 120 comprises a shaker bag and separator 125 comprises a funnel bag, fluid is moved from the shaker bag to the funnel bag and, subsequently, to first waste container 135, if appropriate, using gravity.

Suitable materials for use in any of the apparatus components, including bags and tubing, as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present invention, during at least a single use. The materials are sterilisable by radiation, steam or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers L.L.C., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 115, 115a, 115b, 115c, 115d, 115e, 115f, 115g, 116 and any other valve used in each embodiment may be composed of, but are not limited to, pinch, globe, ball, gate or other conventional valves. In some embodiments, the valves are occlusion valves such as Acro Associates' Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

In an additional embodiment, not shown in FIG. 1, the output fluid in output container 145 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. In embodiments, the sensors are capable of providing such concentration information on a real-time basis and without having to physically transport a sample of the output fluid, or air in the headspace, to a remote device. The resultant modified SAR-CoV-2 particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

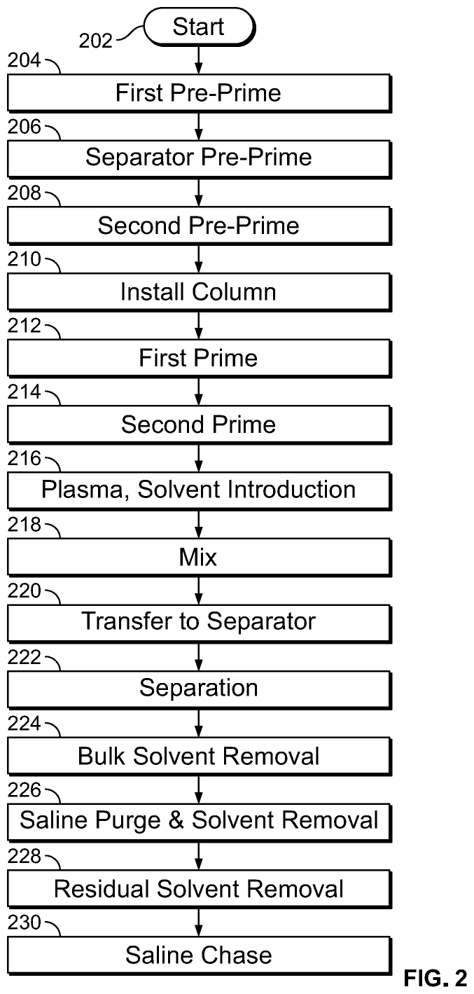

The method of operation of system components 100 of FIG. 1 will now be described in detail below. FIG. 2 is a flow chart illustrating an exemplary process for separating modified SAR-CoV-2 viruses, in accordance with some embodiments of the present specification. The method described in context of FIG. 2 may be implemented using system components 100 described in context of FIG. 1. At 202, a plasma delipidation process is started once the bags and tubing sets are connected in place, as described in FIG. 1. At 204, a first priming fluid pre-primes various fluid lines. In embodiments, fluid lines include the tubing sets and any other channels for transporting the fluids between the system's components.

In some embodiments, the present specification includes a computing device with an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures. In some embodiments, execution of sequences of programmatic instructions enables or causes the processor to perform various functions and processes. In alternate embodiments, hard-wired circuitry may be used in place of, or in combination with, software instructions for implementation of the processes of systems and methods described in this specification. Thus, the systems and methods described are not limited to any specific combination of hardware and software.

In some configurations, the embodiments described in the present specification include a controller having at least a processor or processing circuitry and a system memory that is in data communication with at least one of the basic components of the system of the present specification to control or automate operation of the system, including, but not limited to:

One or more fluid inputs;

One or more mixing devices that may be used to mix fluid from a fluid input and solvent from a solvent input;

One or more valves that may be used to control the flow of a fluid, a solvent or a fluid-solvent mixture;

One or more valves that may be used to control the flow of fluid from a fluid input and to control the flow of solvent from a solvent input;

One or more valves that may be used to control the flow of a fluid-solvent mixture through tubing and to a separator;

One or more separators for performing bulk solvent separation and valves associated therewith;

One or more pressure sensors and/or valves positioned in the flow stream to control the flow of the second layer to the solvent extraction device;

One or more glucose inputs and valves associated therewith;

One or more saline inputs and valves associated therewith;

One or more solvent extraction devices; and/or

One or more pumps, which may be a peristaltic, roller, or rotary pump.

FIG. 3A illustrates a bag 355 containing the priming fluid. In some embodiments, the priming fluid for the first priming is saline. Saline prepares the system by flushing the fluid flow-path between bag 355 and a prime waste container 365. The fluid flow path flushed at this step includes a First Fluid Flow Line (1^{st} FFL) comprising line 380 between bag 355 and a sub-path 321 where sub-path 321 is the fluid flow path between a separator 325 and a prime connector tube 370 (and thus represents a sub-path), a second fluid flow line (2^{nd} FFL) 382 between connector tube 370 and a first pump 350, and a third fluid flow line (3^{rd} FFL) 384 extending between the first pump 350 and second waste container 365. 1^{st} FFL 380 is not in communication with inputs 305, 310, 360, mixer 320, first waste container 335, and output container 345. In embodiments, the first pump 350 functions to draw the priming fluid towards the prime waste or second waste container 365. In embodiments, second waste container 365 is configured to collect the prime waste.

In embodiments, at the preliminary priming stage, a solvent extraction device is separate from the system. The solvent extraction device may be a charcoal column that is subsequently added to the system and used to extract a solvent from plasma that contains modified SARS-CoV-2 particles. The solvent extraction device is substituted with connector tube 370 between the fluid sub-path 321 and 1^{st} FFL 382, between bag 355 and second waste container 365. Pre-priming the fluid lines ensures that air is substantially removed from the fluid lines. Later when the solvent extraction device is connected, the absence of air safeguards the function of the solvent extraction device. In embodiments, the solvent extraction device is a charcoal column comprising coated beads of charcoal. The substantial or material presence of air interferes with the efficiency and surface area of the charcoal column. In embodiments, once the pump is closed, it does not allow for backflow of fluids. At this stage, a first valve 315c, a second valve 315e, and a fifth valve 315g, along 1^{st} FFL are in an open state to facilitate passage and direct the flow of priming fluid from bag 355 to waste container 365 containing the prime waste. Other valves (315, 316, 315a, 315b, fourth valve 315d, 315f, and third valve 330) remain closed to prevent passage of the saline to other parts of the lines, and therefore 1^{st} FFL is not in fluid communication with inputs 305, 310, 360, mixer 320, first waste container 335, and output container 345.

Following step 204, fourth valve 315d and third valve 330 are opened to facilitate passage of priming fluid from bags 360 to separator 325, while the other valves (315, 316, 315a, 315b, 315c, 315e, 315f, and 315g) remain closed. At 206, a priming fluid pre-primes various fluid lines towards a separator 325. FIG. 3B illustrates at least two bags 360 containing the priming fluid. In embodiments, the priming fluid for priming the lines to separator 325 is saline. The saline prepares the system by flushing the lines between bags 360 and separator 325. The fluid flow paths flushed at this step may include line 1^{st} FFL 380 between bags 360 and sub-path 321, and 4^{th} FFL 386 extending from separator 325 to sub-path 321. Therefore, a second fluid flow path (FFP2) may be defined as the path including 1^{st} FFL 380 between bags 360 and sub-path 321, and 4^{th} FFL 386 extending from separator 325 to sub-path 321. FFP2 does not include fluid paths to inputs 305, 310, and 355, mixer 320, prime connector tube 370, pump 350, first waste container 335, second waste container 365, and output container 345. The solvent extraction device is still separate from the system.

Following step 206, fourth valve 315d remains open, second valve 315e and fifth valve 315g are additionally opened, while all other valves (315, 316, 315a, 315b, 315c, 315f, and 330) remain closed in order to facilitate fluid flow along a third fluid flow path (FFP3). FFP3 may be defined as the path of fluid from bags 360, through prime connector tube 370, to second waste container 365. FFP3 is not in fluid communication with inputs 305, 310, and 355, mixer 320 separator 325, first waste container 335, and output container 345. At 208, a second pre-priming operation is performed in the various fluid lines of the system through FFP3. Referring to FIG. 3C, at this stage, priming fluid from bags 360 are transported through fourth valve 315d and second valve 315e, through prime connector tube 370, and through fifth valve 315g, toward second waste container 365, while all other valves remain closed. Step 208 is concluded with presence of priming fluids in the main lines of the system, which include 1^{st} FFL 380, sub-path 321, 4^{th} FFL 386, 2^{nd} FFL 382, and 3^{rd} FFL 384.

Following step 208, the priming fluid transported through the fluid lines is drained into second waste container 365, which is configured to collect prime waste. All the valves (315, 316, 315a, 315b, 315c, 315d, 315f, 315e, 315g, 330) are then closed. Therefore there is no path for flow of fluids. The prime connector tube 370 is clamped and removed from the fluid line. At 210, a solvent extraction device is installed into the system by replacing the prime connector tube 370. Referring to FIG. 3D, a solvent extraction device 340 is installed at the location within the fluid line, between sub-path 321 and 2^{nd} FFL 382, where prime connector tube 370 was originally placed. Fluid lines (sub-path 321, 1^{st} FFL 380, 2^{nd} FFL 382, 3^{rd} FFL 384, and 4^{th} FFL 386) between bags 355 and 360, separator 325, and second waste container 365 are pre-primed, that is, they are primed before installing solvent extraction device 340.

Following step 210, first valve 315c, second valve 315e, and fifth valve 315g are opened while all other valves (315, 316, 315a, 315b, 330, 315d, 315f) remain closed, which defines a fourth fluid flow path (FFP4) extending from input 355, through solvent extraction device 340, to second waste container 365. FFP4 is not in fluid communication with inputs 305, 310, 360, mixer 320, separator 325, first waste container 335, and output container 345. At 212, a first priming is performed of the various fluid lines. Referring to FIG. 3E, priming fluid from bag 355 is depleted and transported through FFP4 comprising first valve 315c and second valve 315e, through solvent extraction device 340, fifth valve 315g, and into second waste container 365. The fluid flow paths that are primed at this step include 1^{st} FFL 380, sub-path 321, 2^{nd} FFL 382, and 3^{rd} FFL 384.

Following step 212, fourth valve 315d is opened, second valve 315e and fifth valve 315g remain open, while all other valves (315, 316, 315a, 315b, 315c, 330, 315f) remain closed, which defines a fifth fluid flow path (FFP5) extending from inputs 360, through solvent extraction device 340, to second waste container 365. FFP5 is not in fluid communication with inputs 305, 310, 355, mixer 320, separator 325, first waste container 335, and output container 345. At 214, a second priming is performed of the various fluid lines through FFP5. Referring to FIG. 3F, priming fluid from bags 360 is transported through fourth valve 315d, second valve 315e, through solvent extraction device 340, fifth valve 315g, and into second waste container 365. The fluid flow paths that are primed at this step include sub-path 321, 1^{st} FFL 380, 2^{nd} FFL 382, and 3^{rd} FFL 384.

At the conclusion of steps 212 and 214, all main fluid lines including sub-path 321, 1^{st} FFL 380, 2^{nd} FFL 382, and 3^{rd} FFL 384, separator 325 and solvent extraction device 340 are primed. In embodiments, fluid lines extending from bottom of separator 325 to second waste container 365, configured to contain prime waste, is filled with priming fluid. Priming also results in removal of small particulates from solvent extraction device 340.

Following step 214, valve 315 is opened and all other valves (316, 315a, 315b, 315c, 315d, 315e, 315f, 315g, and 330) are closed, defining a sixth fluid flow path (FFP6) from input 305 to mixer 320. FFP6 is not in fluid communication with bag, 310, separator 325, first waste container 335, inputs 310, 355, 360, solvent extraction device 340, pump 350, second waste container 365, and output container 345. At 216, the plasma fluid and the solvent are introduced one after the other, into a mixing device 320 of the system. In embodiments, a blood fraction of the patient is obtained, which in a still further embodiment is plasma. The process of blood fractionation is typically achieved by filtration, centrifuging the blood, aspiration, or any other method known to persons skilled in the art. Blood fractionation separates the plasma from the blood. In an embodiment, blood fractionation is performed remotely. In one embodiment, blood is withdrawn from a patient in a volume sufficient to produce about 12 ml/kg of plasma based on body weight. During the fractionation process, the blood can optionally be combined with an anticoagulant, such as sodium citrate, and centrifuged at forces approximately equal to 2,000 times gravity. The blood is separated into plasma and red blood cells using methods commonly known to one of skill in the art, such as plasmapheresis. In an embodiment, the red blood cells are then aspirated from the plasma. In one embodiment, the process of blood fractionation is performed by withdrawing blood from the patient infected with SARS-CoV-2, and who is being treated by the physician. In an alternative embodiment, the process of blood fractionation is performed by withdrawing blood from a person other than the patient infected with SARS-CoV-2 who is treated by the physician. Therefore, the plasma obtained as a result of the blood fractionation process may be either autologous or non-autologous.

Subsequent to fractionation, the red blood cells are either stored in an appropriate storage solution or, preferably, returned to the patient during plasmapheresis. Physiological saline, 5% albumin, or other suitable fluid may also optionally be administered to the patient to replenish volume. If the blood was obtained from an individual other than the patient, the cells are returned to that individual, who can also be referred to as the donor.

Plasma obtained from blood is usually a straw-colored liquid that comprises the extracellular matrix of blood cells. Plasma is typically 95% water, and contains dissolved proteins, which constitute about 6-8% of plasma. The plasma also contains glucose, clotting factors, electrolytes, hormones, carbon dioxide, and oxygen. The plasma has a density of approximately 1006 kg/m3, or 1.006 g/ml.

In some alternate embodiments, SARS-CoV-2 viral particles are separated from the plasma. In embodiments, autologous or non-autologous plasma collected from the patient or donor, respectively, is subsequently treated via an approved plasmapheresis device. The plasma may be transported using a continuous or batch process.

Referring to FIG. 3G, a plasma input bag 305 contains the plasma that will be treated by the various embodiments of the present specification. The plasma is transported from bag 305, along FFP6, through a valve 315, into mixing device 320.

Following transportation of plasma from bag 305 to mixing device 320, valve 315 is closed and valve 316 is opened, while all the other valves (315a, 315b, 315c, 315d, 315e, 315f, 315g, 330) remain closed, thus defining a seventh fluid flow path (FFP7) from bag 310 to mixing device 320. FFP7 is not in fluid communication with bag 305, separator 325, first waste container 335, inputs 310, 355, 360, solvent extraction device 340, pump 350, second waste container 365, and output container 345. Referring to FIG. 3H, a solvent input bag 310 contains the solvent. The solvent is transported along FFP7, from bag 310, through valve 316, into mixing device 320. The solvent is used for extracting lipids from the plasma fluid or from particles within the plasma fluid. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1,1,3,3,3-hexafluoro-2-(fluoromethoxy) propane-d3), perfluorocyclohexanes, trifluoroethane, cyclofluorohexanol, and combinations thereof. In an embodiment, a mix of Sevoflurane and n-butanol is used as the solvent. In an embodiment a volume ratio of Sevoflurane and n-butanol used is 95:5. In various embodiments, the plasma and the solvent are transported in any order. While transporting the plasma and the solvent to mixing device 320, the valves corresponding to bags containing the plasma (valve 315) and the solvent (valve 316) are respectively open. All other valves (315a, 315b, 315c, 315d, 315e, 315f, 315g, and 330) remain closed.

Following step 216, once the plasma and the solvent are in mixing device 320, all the valves (316, 316, 315a, 315b, 315c, 315d, 315e, 315f, 315g, 330) are closed. At 218, the plasma and the solvent, which are present together within the mixing apparatus are processed by a mixing operation. In an embodiment, the solvents used include either or both of organic solvents sevoflurane and n-butanol. In embodiments, the solvent is optimally designed such that only the SARS-CoV-2 viral particles are treated to reduce their lipid levels.

The mixing process includes factoring in variables such as the solvent employed, mixing method, time, and temperature. Choice of a mixing method may also affect some system requirements, such as but not limited to functional requirements, packaging requirements, cost, and environmental requirements. Additional variables that may be considered in system design are as follows:
1. Priming. Whether the system can handle priming so that it can be primed and ready for a process.
2. Continuous or Interrupted Flow. Whether the system has to be continuous in nature (continuously separates plasma and inputs replacement fluid in parallel) or can handle discrete amounts of liquid (batch flow).
3. Closed Loop Control. Whether the system has to be monitored or can be validated by process.
4. Variable Flow. Whether the system can handle various ranges of flow.
5. Hold-up Volume. This is the amount of fluid or blood that remains in the circuit after the process is complete, as it is desirable to have as little blood or fluid outside of the patient at any given time and so that the plasma that will be returned to the patient is not overdiluted.
6. Mixing Control. Whether the system allows for controlling the level, speed or extent
of mixing. 7. Plasma Range. Whether the system can handle different types of plasma, including normal plasma, high lipid plasma, high triglyceride plasma, and other types of plasma.
8. Packaging Requirements. Whether a hospital or blood bank could accommodate the footprint of the system and its associated components, including disposables and hardware.
9. Environmental Requirements. Whether the system could be deployed in a variety of settings with respect to operating noise/vibration and hardware durability (for example, whether it can be deployed in bloodbanks or hospitals).
10. Cost. Whether the system can be manufactured in a cost-effective manner (for example, no high cost, high precision connectors).

Different mixing methods provided different results in terms of remaining SAR-CoV-2 viral particles and native blood constituents within the delipidated plasma obtained after the mixing. The mixing methods employed in the present specification take into account a plurality of variables that, when combined, achieve an ideal mixing environment for optimal selective delipidation. By way of background, several mixing methods, as are well-known to those of ordinary skill in the art were initially employed with little to no success. These methods included continuous vortex mixing, mixing using a static mixer, mixing using a silly straw mixer, and mixing using a rotating cylinder.

Continuous vortex type of mixing involves using a vortexer to mix smaller quantities of liquid. When a test tube or other container is pressed into the rubber cup of the vortexer, the motion is transferred to the liquid inside, creating a fluid vortex or whirlpool in an off-center rotation. Because the speeds achieved are close to 2500 rpm, the end result could be “overdelipidation”, or complete delipidation of SARS-CoV-2.

A static mixer is a plate-type mixer or a mixer comprising mixing elements contained within an elongated housing that effectuates movement of a tube containing a mixture or mixture of fluids, where the movement is typically sideways from one side to another. It is typically employed for continuous mixing. This method of mixing does not work as it 1) involves direct connection to the patient for serial apheresis and delipidation and also results in “overdelipidation”, or complete delipidation of SARS-CoV-2.

The “silly straw” method of mixing was designed as a coiled tube (a tubing set wrapped around a stick) through which a fluid mixture flows continuously creating a Taylor vortex. A continuous flow of plasma and solvent in a 2:1 solvent to plasma ratio was used. This method proved to be entirely ineffective. One theory is that in order to effectively selectively delipidate, the plasma and solvent mixture needs to be in full contact at a specified ratio, for a specified amount of time and that an instantaneous flow-through process could not achieve this equilibrium.

Mixing using a rotating cylinder involves a tube containing the mixture that rotates around its axis (similar to the movement of a record on a turntable) to mix the fluids within the tube. In using this process, the fluid tumbles from the top to the bottom of the test tube. While the method was not optimal for selective delipidation as described in the present specification, a novel mixing bag of specific geometry and size was designed and implemented.

The system of the present specification, and in particular, the mixing sub-system is designed so that it can be primed and ready for a process. In addition, the system of the current specification can handle fluid processing in batches without the need for continuous flow. The system of the present specification advantageously does not require closed loop control. Once the parameters (flow rate, volume) are established, the system is gravity based and operates accordingly. Optionally, a charcoal column is employed to further ensure that all solvent is removed. The system of the present specification can also accommodate various ranges of fluid flow. Because the system of the present specification is a stand-alone system (meaning that apheresis is not integrated), the issue of hold-up volume becomes a non-issue. The system of the present specification also allows for controlling the level of mixing by determining the speed of the mixer and using a mixing bag with an appropriate volume and geometry. Further, the system of the present specification is designed to be able to handle a wide and infinite range of plasma that can be treated by the system, including, but not limited to normal plasma, high lipid plasma, high triglyceride plasma, and other types of plasma. The system of the present specification has low to minimal footprint, is readily and easily deployable in a variety of environments with minimal noise impact. In addition, the system of the present specification can be manufactured in a cost-effective manner.

Referring to FIG. 3I, mixing device 320 may be a bag used for mixing the plasma and the solvent. In embodiments, mixing device 320 includes both an orbital mixer and a mixing bag, and is placed at an angle within the system. In one embodiment, the mixing bag is placed horizontally over the orbital mixing device, because this position may receive the most optimal orbital mixing action for the fluid contained in the bag, as most of the fluid would gravitate towards the bottom. The bag and its corners can be used to impart energy. The mixing bag, using an angled platform as described below may be placed at a slight angle to enable draining of the fluids. The angle may range from 0 degrees (completely vertical) to 90 degrees (completely horizontal). In one embodiment, the platform upon which the mixing bag rests is placed at an angle of 18.2 degrees. In one embodiment, mixing device 320 is of a circular shape. In another embodiment, mixing device 320 is of a rectangular shape.

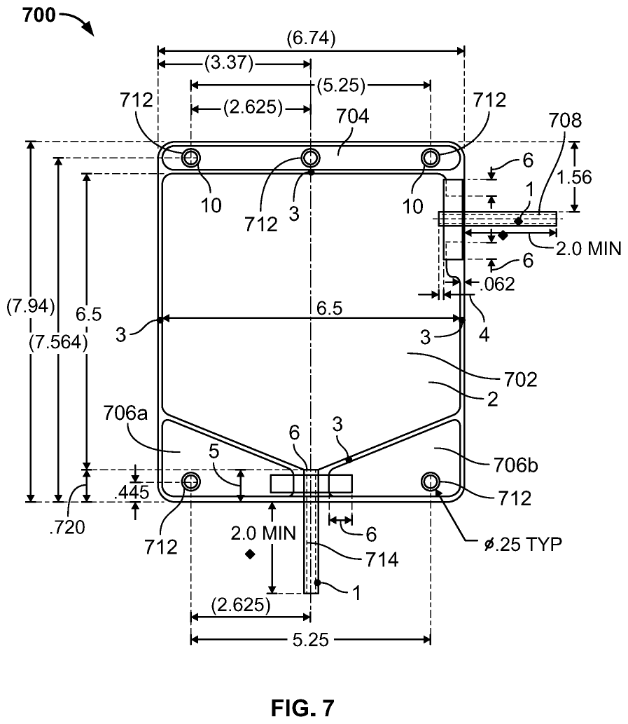

FIG. 7 illustrates an exemplary mixing device (bag) 700, in accordance with embodiments described in context of FIG. 3I. Bag 700 is of a rectangular shape, comprising a section 702 where plasma and solvent fluid may be received through an input pipe 708. Section 702 has at least five edges and is at the center of bag 700. Section 702 is surrounded by sealed sections of bag 700. One of the edges of bag 700 includes a rectangular sealed section 704. Two mutually inclined edges, opposite to the edge along section 702, include triangular sealed sections 706a and 706b. Each sealed section 704, 706a, and 706b, includes at least one hanger hole, such as holes 712. Section 702 includes an output pipe 714 positioned between triangular sealed section 706a and 706b, to enable letting out of the mixture.

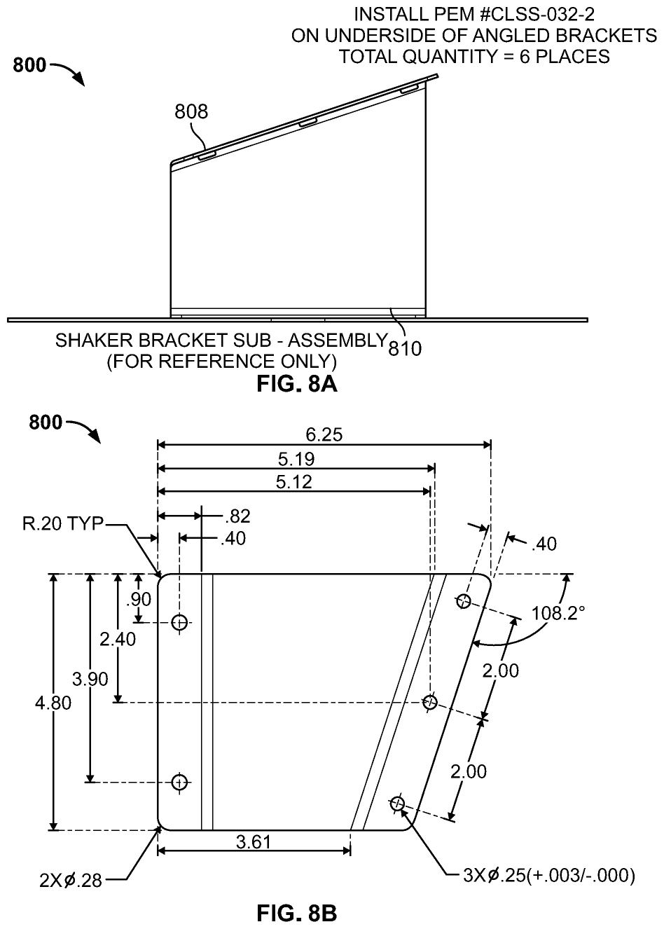

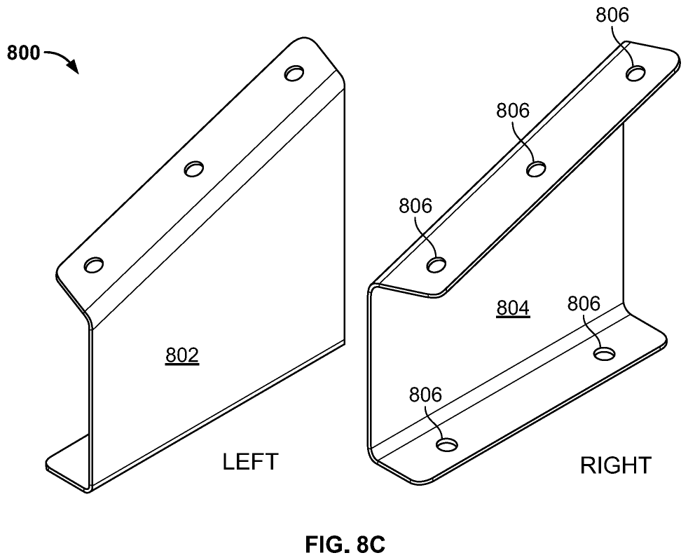

FIG. 8A illustrates a side view of shaker angle brackets 800 that are used to position mixing device 320 within the system, in accordance with some embodiments of the present specification. FIG. 8B illustrates a another side view of shaker angle brackets 800 that are used to position mixing device 320 within the system, in accordance with some embodiments of the present specification. FIG. 8C illustrates a perspective view of shaker angle brackets 800 that are used to position mixing device 320 within the system, in accordance with some embodiments of the present specification. Referring simultaneously to FIGS. 8A, 8B, and 8C, the mixing bag used to perform the mixing operation may be placed over an orbital mixer, which is fitted within the system with the help of brackets 800. In embodiments, brackets 800 are manufactured from Aluminum. In one embodiment, the Aluminum used for making brackets 800 is 0.060 inches thick. Referring simultaneously to FIGS. 8A, 8B, and 8C, brackets 800 include two parts 802 and 804, which mirror each other. In one embodiment, bracket 802 is placed on the left and bracket 804 is placed opposite to bracket 802 on the right. A mixing device, such as device 320 of FIG. 3I, is positioned on brackets 802 and 804. Holes 806 on both brackets 802 and 804 enable fixing the mixing device. In one embodiment, the mixing device provides a platform for placing the mixing bag, such as bag 700 of FIG. 7. Each bracket has two opposing sides connected by a flat surface between them. A top side 808 of each bracket is inclined at an angle for placing the mixing bag. In embodiments, the angle is in the range of 0 to 90 degrees. In one embodiment, the angle is 18.2 degrees, relative to a horizontal bottom side 810. The incline provided by top side 808 enables placing the mixing device, and therefore the mixing bag at an inclination, for an optimal mixing operation. Each edge 808 and 810 is bent in two ways to created angled brackets 802 and 804. The bent portions include holes 806 that enable fixing the mixing device with brackets 800.

In one embodiment, mixing device 320 has a capacity of 300 milliliters (ml). In one embodiment, mixing device 320 is configured to mix approximately 100 ml of plasma with the solvent during a single mixing operation. In one embodiment, solvent and plasma are mixed in a volume ratio of 2:1. For example, 100 ml of plasma is mixed with 200 ml of the solvent. In another embodiment, the solvent and plasma are mixed in a volume ratio of 1:1.

Solvent type, ratios and concentrations may vary in this step. Acceptable ratios of solvent to plasma include any combination of solvent and plasma. In some embodiments, (volume) ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 5% n-butanol in the final solvent/plasma mixture. In an embodiment, a final concentration of n-butanol in the final solvent/plasma mixture is 3.33%. In embodiments, the final concentration of n-butanol in the resultant solvent/plasma mixture may vary and may be dependent on the solvent to plasma ratio, which may also vary. The plasma may be transported to the mixing device using a continuous or batch process. Further various sensing means may be included to monitor pressures, temperatures, flow rates, solvent levels, and the like. The solvents dissolve lipids from the plasma. In embodiments of the present specification, the solvents dissolve lipids to yield treated plasma that contains modified SARS-CoV-2 viral particles with reduced lipid content. It should be noted that there is no clinically significant decrease in desired, native blood constituents post-plasmapheresis.

In one embodiment, mixing device 320 is operated to mix the plasma solvent mixture for 60 seconds with an average mixing plasma batch volume of 99±7.5 ml.

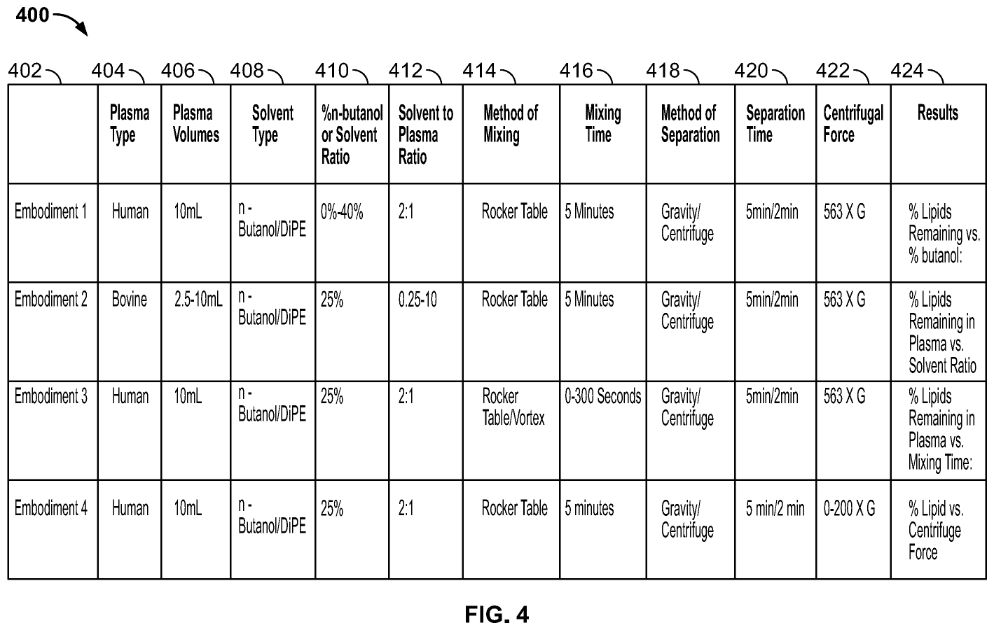

In various embodiments, various energy measurements are provided as input to operate mixing device 320. Energy is introduced into the system in the form of varied mixing methods, time, and speed. A combination of the mixing parameters such as but not limited to the volume ratio of solvent to plasma, shape of the mixing device 320, and the amount of energy input used to operate mixing device 320, directly affect the success of the mixing operation to achieve delipidated SARS-CoV-2 viral particles in the plasma. In one example, a solvent to plasma ratio of 2:1, for a batch of 100 ml plasma, mixed for 60 seconds, in a rectangular mixing device, using an energy input of 200 RPM does not delipidate the SARS-CoV-2 viral particles from the plasma. In another example, a solvent to plasma ratio of 2:1, for a batch of 100 ml plasma, mixed for 60 seconds, in a large square mixing device, using an energy input of 400 RPM also does not delipidate the SARS-CoV-2 viral particles from the plasma. Therefore, multiple parameters affect the success of delipidating SARS-CoV-2 viral particles from the plasma. The effect of varying the different parameters is described in the subsequent sections, and in context of experiments illustrated in FIGS. 4, 5, and 6.

Referring back to step 218 of FIG. 2, and FIG. 3I, the plasma and the solvent interact with each other within mixing device 320 to the extent that SARS-CoV-2 viral particles are delipidated, while other blood constituents are not. The process is therefore termed as selective delipidation. In embodiments, the mixing is performed in order to achieve at least 20% to 99%, and every increment therein, delipidation of SARS-CoV-2 viral particles.

After the mixing, valve 315a is opened while all the other valves (315, 316, 315b, 315c, 315d, 315e, 315f, 315g, 330) remain closed, thus defining an eighth fluid flow path (FFP8) between mixing device 320 and separator 325. FFP8 is not in fluid communication with bags 305, 310, 355, 360, waste containers 335, 365, solvent extraction device 340, pump 350, and output container 345. At 220, once the mixing operation is completed, the solvent plasma mixture is transferred to a separator along FFP8, where the plasma and the solvent are separated by gravity. Referring to FIG. 3J, mixture of solvent and plasma is dropped down through a valve 315a into a separator 325. The mixture remains in separator 325 until the solvent settles at the bottom of separator 325. The plasma is separated and remains in a layer above the solvent. The solvent employed is preferably of a higher density than plasma, and therefore settles at the bottom.

In embodiments, steps 216, 218, and 220 are performed iteratively, in batches, until separator 325 is filled to its capacity. Once the separator is filled to its capacity, all the valves (315, 316, 315a, 315b, 315c, 315d, 315e, 315f, 315g, 330) are closed. At 222, and referring to FIG. 3K, the collected mixture of plasma and solvent in separator 325 is allowed to stand for a period of time, until the solvent separates and settles at the bottom of separator 325. In embodiments, the period of time is dependent upon the time it takes for the solvent to fully separate without a loss or sacrifice in the amount of plasma. In one embodiment, the mixture is allowed to stand for approximately 30 minutes. In embodiments, separator 325 has a cone-shaped bottom that enables easy removal of bulk solvent in a subsequent step.

Following the separation, valve 315b is opened to define a ninth fluid flow path (FFP9) from separator 325 to first waste container 335. FFP9 is not in fluid communication with bags 305, 310, 355, 360, mixer 320, second waste container 365, solvent extraction device 340, pump 350, and output container 345. At 224, bulk solvent is removed from the separator 325 along FFP9. Referring to FIG. 3L, bulk solvent that has settled at the bottom portion of separator 325 flows to a first waste container 335 through a valve 315b, which, when open allows fluid to flow freely using gravity. In other embodiments, a pump may be employed to remove the solvent. Cone-shaped bottom of separator 325 aids easy removal of bulk solvent. Valve 315b is closed after bulk solvent has been moved through it and before the plasma from separator 325 reaches valve 315b.

In embodiments, a weight of separator 325 is known, in addition to weight of the plasma and of the solvent. In embodiments, the weight of the separator bag is continuously monitored. With this information, valve 315b is closed as soon as the amount of solvent removed from separator 325 corresponds to the known weight of the solvent. The weight of the solvent that flows to the first waste container 335 is, in an embodiment, indirectly monitored, because the amount of solvent that is added to the system and the amount of solvent present in the separator waste bag are known. In addition, the residual concentration of solvent that is in the plasma is based on validation of system parameters and a validated analysis of residual solvent concentrations via GC over many process runs.

Once valve 315b is closed, valves 330, 315e, and 315g are opened while all other valves (315, 316, 315a, 315c, 315d, 315f, 330) remain closed, thus defining a tenth fluid flow path (FFP10). FFP10 is not in fluid communication with bags 305, 310, 355, 360, mixing device 320, first waste container 335, second waste container 365, and output device 345. At 226, and referring to FIG. 3M, a pump 350 is turned on and valves 330 and 315e are opened in order to pull plasma from separator 325 through fluid sub-path 321 towards solvent extraction device 340, along FFP10. During this operation, initially fifth valve 315g is simultaneously open. Valve 315g is placed between solvent extraction device and second waste container 365. As the pump pulls the fluid present in sub-path 321 from separator 325 through solvent extraction device 340, priming fluid that was initially present in sub-path 321, 2^{nd} FFL 382, and 3^{rd} FFL 384, extending between separator 325 and second waste container 365, is pushed, or chased, further ahead in the lines by the plasma being pulled through fifth valve 315g, towards second waste container 365 configured to contain prime waste. Once plasma (pulled by pump 350) reaches fifth valve 315g, which is determined using both the tube length and the volume of fluid passed through the pump per revolution, the fifth valve 315g is closed so that priming fluid is separated from plasma. This ensures that the plasma is not diluted and additional fluids are not collected along with the plasma that will subsequently be delivered back to the patient.

Subsequently, valve 315f is opened in addition to already open valves 330, 315e, while all other valves (315, 316, 315a, 315b, 315c, 315d, 315g) remain closed, thus defining another fluid flow path (FFP11) from separator 325, through solvent extraction device 340, to output device 345. At 228, and referring to FIG. 3N, pump 350 further pulls plasma along FFP11, from separator 325 through valves 330 and 315e, through solvent extraction device 340, and through a valve 315f, towards and into an output plasma container 345. As plasma moves through solvent extraction device 340, charcoal in solvent extraction device 340 absorbs and therefore extracts any remaining solvent from the plasma.

After extracting the delipidated plasma in to output container 345, valve 330 is closed along with valves 315, 316, 315a, 315b, 315c, 315g, and fourth valve 315d is opened along with open valves 315e and 315f, thus defining another fluid flow path FFP12 from bags 360 through solvent extraction device 340, to output device 345. FFP 12 is not in fluid communication with bags 305, 310, 355, mixing device 320, separator 325, first waste container 335, and second waste container 365. At 230, and referring to FIG. 3O, once plasma is pulled out from separator 325 completely, pump 350 is still operated until priming fluid from bags 360 along FFP12 to follow or chase the plasma in the 1^{st} FFL 380, sub-path 321, 2^{nd} FFL 382, and 3^{rd} FFL 384, through second valve 315e, through solvent extraction device 340, and through valve 315f. Pump 350 is stopped once the priming fluid, chasing the plasma, reaches a position in the fluid line which is just before reaching output plasma container 345. In an embodiment, 150 mL of priming fluid is used to further chase the plasma into the plasma output bag 145/345 to ensure full recovery of the delipidated plasma. In an embodiment, chasing the fluid flow to the prime waste occurs to the point where pump 350 reaches a specific number of revolutions that is indicative of the plasma volume that has flowed through the system. Thus, the revolutions of the pump control how much fluid is in the prime waste or second waste container 345. Pump 350 is stopped to ensure that as much of the delipidated plasma that is available in the system is collected in container 345, while the collected plasma is saved from unnecessary dilution by priming fluids. In embodiments, pump 350 is stopped automatically by the system based on the amount of plasma collected, which corresponds to the known amount of input plasma. In embodiments, the configurations of the disposable elements within the system are employed to program the system to automatically stop pump 350. In an embodiment, the tubing sets are of a known length and diameter. In addition, the volume of solvent at the bottom of separator bag 325 is also known in addition to the amount of plasma.

At the end of this process, solvent waste is collected separately in first waste container 135/335, and prime waste is collected in second waste container 165/365, through their separate waste streams. This is advantageous for many reasons. Primarily, it is more expensive to dispose of certain types of waste, such as solvent waste. If solvent waste is “contaminated” with or combined with other types of waste, the additional waste will have to be disposed of in the same costly manner as solvent waste. Prime waste, for example, which consists of mostly saline and/or glucose, can be directed to a normal hospital waste stream. If mixed with solvent, the prime waste will have to be diverted into the chemical waste disposal channels, by default. By separating out waste, each waste stream can be treated and disposed of appropriately. In some embodiments, the solvent waste can be treated or scrubbed to reclaim a pure solvent so that it can be re-used.

Examples of effect of varying multiple parameters are now explained briefly. Among various methods by which plasma may be delipidated, parameters affecting the extent of delipidation may be broadly identified as chemical and mechanical parameters. Examples of chemical parameters may include, but may not be limited to, plasma type (bovine, human, lipemic), plasma volumes, solvent type (n-butanol/DiPE or n-butanol/sevoflurane, any other), percentage of n-butanol present in the solvent, and solvent to plasma ratio. Examples of some of the mechanical parameters may include, a method of mixing (rocker table, vortex, any other), mixing duration, method of separation (gravity, centrifuge, any other), separation time, and centrifuge force.

For purposes of illustrating the effect of varying these parameters on the extent of delipidation, an experimental delipidation process was performed in a laboratory setting. The results, presented in table 400 of FIG. 4, are briefly discussed herein. Referring to table 400, first column 402 lists different embodiments in separate rows. Each embodiment corresponds to a unique combination of the parameters that affect the extent of delipidation. The second column 404 lists the plasma type used for each embodiment. The plasma type was selected from the plasma of a human or that of a bovine. Column 406 lists the plasma volumes (in milliliter) used in each embodiment. Column 408 lists the type of solvent used. In most embodiments, the solvent type is either of n-Butanol and DiPe. Column 410 lists the percentage of n-Butanol used, which may also be inferred as an indication of the solvent ratio. Column 412 lists the solvent to plasma ratio used in each embodiment. Column 414 lists the type of mixing method used for each embodiment. Column 416 lists the time for which the mixing process was implemented.

Column 418 lists the chosen method for separation of the plasma and the solvent. Column 420 lists the time for which the process of separation was performed for each embodiment. Column 422 lists the amount of centrifugal force applied for each embodiment. Lastly, column 424 lists the results that show the variation across each embodiment, in the percentage of lipids that remain in the treated plasma.

Embodiment 1: About 10 milliliters (ml) of plasma derived from a human was used. This plasma was mixed with n-butanol/DiPE solvent. A solvent to plasma ratio of 2:1 was used. The rocker table was used to perform the mixing operation, for about five minutes. A centrifugal force of 563×G was applied for about two minutes to separate the delipidated plasma from the solvent. The effect of varying percentage of n-butanol in the solvent within a range of 0% to 40% is that remaining lipids progressively decrease with an increase in the quantity of n-butanol in the solvent.

Embodiment 2: In another similar experiment, 10 ml of bovine plasma was mixed with n-butanol/DiPE solvent containing 25% n-butanol. The mixture was mixed using a rocker table for about five minutes. A centrifugal force of 563×G was applied for about two minutes to separate the delipidated plasma from the solvent. The effect of varying the solvent to plasma ratio in a range of 0.25 to 10 is that a lower ratio, specifically within a range of 1 to 2, results in most reduced lipid concentration in the delipidated solution.

Embodiment 3: In another similar experiment, 10 ml of human plasma was mixed with n-butanol/DiPE solvent containing 25% n-butanol, using a solvent to plasma ratio of 2:1. Different samples of the mixture were mixed using a rocker table and using a vortex. Gravity separation was used for about five minutes to separate some of the samples, as well as a centrifugal force of 563×G was applied for about two minutes to separate the delipidated plasma from the solvent for the remaining samples. The effect of different mixing methods and by varying the duration of mixing for both the methods used for separating (gravity and centrifuge) is that there is a variation on the concentration of lipids remaining in the delipidated plasma.

Embodiment 4: In yet another similar experiment, 10 ml of human plasma was mixed with n-butanol/DiPE solvent containing 25% n-butanol, using a solvent to plasma ratio of 2:1. The mixture was mixed using a rocker table for about five minutes. A range of centrifugal force was applied for about two minutes to separate the delipidated plasma from the solvent. The effect of varying the centrifugal force used for separation on the lipid concentration remaining in the delipidated plasma

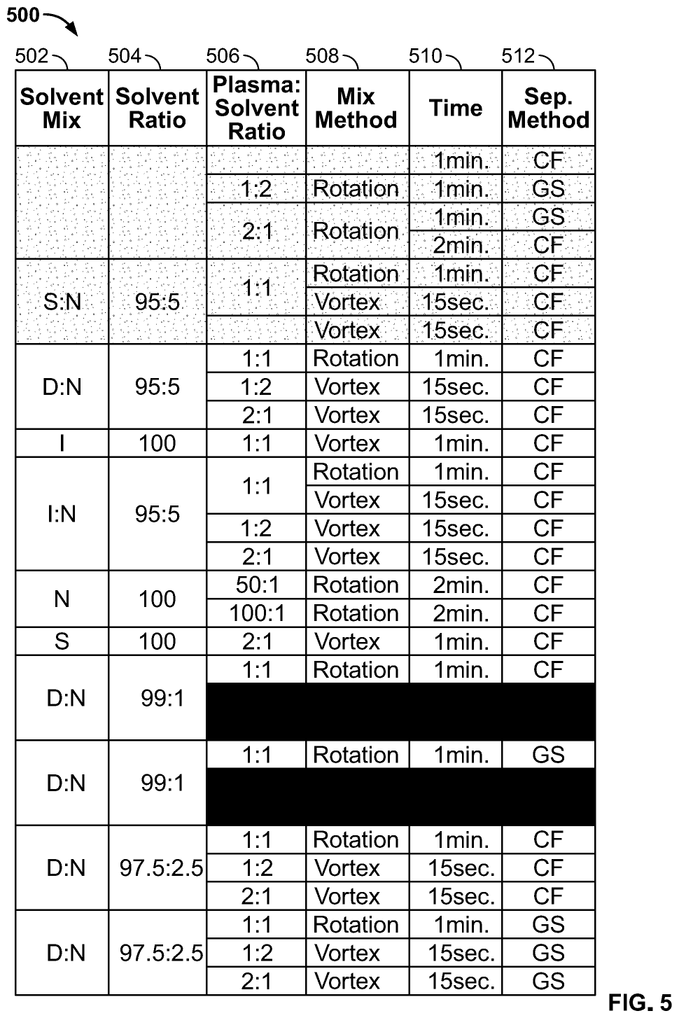

FIG. 5 is a table 500 that lists another exemplary set of variables that may affect the delipidation process and resultant percentage selective delipidation and is presented by way of example only to show possible combinations of variables. Any combination of variables may be employed as long as they achieve the objectives of the present specification. The ideal results from these experiments include a substantial change in SARS-CoV-2 viral concentration, no change in other native blood constituents, including preservation of Apo-A1, preservation of Apo-B, and preservation of phospholipids, resulting in selective delipidation of SARS-CoV-2 viral particles. Referring to the table 500, the first column 502 lists the type of solvent mix used. The constituents for the solvent solution may contain one or more of Sevoflurane (S), n-butanol (N), DiPE (D), and Isofluorane (I). The second column 504 (Solvent Ratio) lists the ratio of the constituents of the solvent that may be used in the solvent solution, corresponding to the first column. The third column 506 (Plasma: Solvent Ratio) lists the proportion of the plasma and the solvent that may be mixed together for the delipidation. The fourth column (Mix Method) 508 states the corresponding mixing method that may be used. The fifth column (Time) 510 provides the corresponding duration for which mixing can be performed. The sixth column (Sep. Method) 512 lists the method used for separation of the delipidated plasma and the solvent. The two methods commonly used for separation are gravity separation (GS) and centrifugal separation (CF), in the embodiments of the present specification. In embodiments, it should be noted that any combination and sub-combination of the solvent mix 502, the solvent ratio 504, the plasma:solvent ratio 506, the mixing method 508, the time 510, and the separation method 512 may be used as long as they achieve the objectives of the present specification.

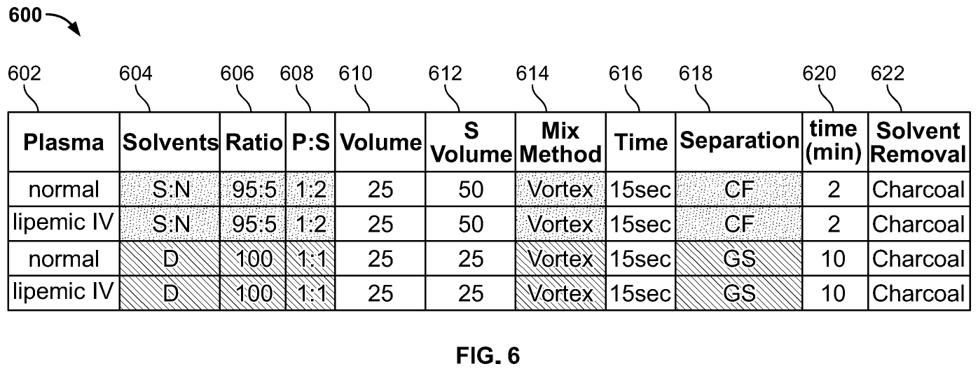

FIG. 6 is a table 600 that provides another exemplary set of variables that may be used for normal plasma and lipemic IV plasma using different solvents and different methods of separation. A first column 602 (Plasma) lists the type of plasma (normal or lipemic IV) used in each embodiment, where each row corresponds to a different embodiment. Column 604 (solvents) lists the type of solvent or solvent mixture used corresponding to each embodiment. Column 606 (Ratio) lists the ratios of constituents in a solvent mixture for each embodiment. Column 608 (P:S) lists the plasma to solvent ratio corresponding to each embodiment. Column 610 (Volume) lists the volume of the plasma used for each embodiment. Column 612 (S Volume) lists the volume of the solvent/solvent mixture used for each embodiment. The volumes of the plasma and the solvent/solvent mixture corresponds to the ratio listed in column 608. Column 614 (Mix Method) lists the method of mixing used for each embodiment. Column 616 (Time) lists the duration for which the mixing was performed. Column 618 (Separation) lists the method used for separation (centrifugal or gravity separation) of the plasma and the solvent. Column 620 (time(min)) lists the duration (in minutes) for which the separation process was performed for each embodiment. Lastly, column 622 (Solvent Removal) lists the type of method used for solvent removal. As seen in table 600, a charcoal column was used in all the embodiments to remove the solvent.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

To enable the operation of the above-described embodiments of the present invention, it is preferable to supply a user of such embodiments with a packaged set of components, in kit form, comprising each component required to practice embodiments of the present specification. The kit may include an input fluid container, a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of generating a composition for treating a patient having a coronavirus viral load greater than a predefined threshold, comprising:
acquiring plasma from the patient;
using a plasma delipidation system, mixing the plasma with an extraction solvent to delipidate at least some of the viruses of the coronavirus viral load, wherein the delipidation of the at least some of the viruses of the coronavirus viral load causes one or more modifications in each of the at least some of the viruses of the coronavirus viral load to form delipidated viruses of the coronavirus viral load;
removing the extraction solvent from the plasma with the at least some of the delipidated viruses of the coronavirus viral load; and
administering the plasma with the at least some of the delipidated viruses of the coronavirus viral load to the patient.

2. The method of claim 1, wherein a volume of the plasma is at least 0.5 liters.

3. The method of claim 1, further comprising repeating each step of claim 1 once per day until the coronavirus viral load of the patient is below a second predefined threshold level.

4. The method of claim 1, further comprising repeating each step of claim 1 once per day, and no more than three times in a week, until the coronavirus viral load of the patient is below a second predefined threshold level.

5. The method of claim 1, further comprising repeating each step of claim 1 once per day, and no more than three times in a week, until the coronavirus viral load of the patient is below a second predefined threshold level and a level of the delipidated viruses of the coronavirus viral load is above a third predefined threshold level.

6. The method of claim 1, further comprising repeating each step of claim 1 once per day for a period of 3 to 7 days.

7. The method of claim 1, wherein the coronavirus is SARS-CoV-2 or variants thereof.

8. The method of claim 7, wherein the delipidation process is warranted if the patient's viral load of SARS-CoV-2 or variants thereof is equal to or greater than 9×10\hat{ }10 viral copies per liter of plasma.

9. The method of claim 8, wherein if the patient's viral load of SARS-CoV-2 or variants thereof is less than 9×10\hat{ }10 viral copies per liter of plasma, the viral load may be increased by introducing live or inactivated, but not denatured virus to the extracted plasma prior to subjecting the plasma to the delipidation process.

10. The method of claim 8, wherein if the patient's viral load of SARS-CoV-2 or variants thereof is less than 9×10\hat{ }10 viral copies per liter of plasma, the plasma delipidation process may be altered to delipidate both the virus and high-density lipoproteins in extracted plasma using at least one solvent mixture.

11. The method of claim 7, wherein at least one of the one or more modifications comprises exposing or removing lipids from one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

12. The method of claim 7, wherein at least one of the one or more modifications comprises modifying a three-dimensional conformational configuration of the target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

13. The method of claim 7, wherein at least one of the one or more modifications comprises removing lipid covering one or more epitopes positioned adjacent to one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

14. The method of claim 7, wherein at least one of the one or more modifications comprises increasing a surface accessibility of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

15. The method of claim 7, wherein at least one of the one or more modifications comprises changing a three-dimensional conformational structure of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

16. The method of claim 7, wherein at least one of the one or more modifications comprises decreasing a lipid content of one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

17. The method of claim 7, wherein at least one of the one or more modifications comprises modifying a physical position one or more of a target set of epitopes relative to another one of the target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-2 including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

18. The method of claim 7, wherein at least one of the one or more modifications comprises modifying a receptor binding domain of each of the SARS-CoV-2 viruses by decreasing an amount of lipid surrounding one or more of a target set of epitopes in each of the SARS-CoV-2 viruses, wherein the target set of epitopes comprises at least one of KYFKNHTSP, TTKR, YYHKNNKSWM, ASTEK, AWNRKR, EQDKNTQ, GTNTSN, KYNENGT, LDSKTQ, PKKS, YQTQTNSPRRAR, TKRT, DEDDSE, GYQPYRVVVL, QPYRVVVLSF, PYRVVVLSF, SARS-CoV-derived T cell epitopes identical in SARS-CoV-2 including one or more of ILLNKHID, AFFGMSRIGMEVTPSGTW, MEVTPSGTWL, GMSRIGMEV, ILLNKHIDA, ALNTPKDHI, IRQGTDYKHWPQIAQFA, KHWPQIAQFAPSASAFF, LALLLLDRL, LLLDRLNQL, LLNKHIDAYKTFPPTEPK, LQLPQGTTL, AQFAPSASAFFGMSR, AQFAPSASAFFGMSRIGM, RRPQGLPNNTASWFT, YKTFPPTEPKKDKKKK, GAALQIPFAMQMAYRF, MAYRFNGIGVTQNVLY, QLIRAAEIRASANLAATK, FIAGLIAIV, ALNTLVKQL, LITGRLQSL, NLNESLIDL, QALNTLVKQLSSNFGAI, RLNEVAKNL, VLNDILSRL, or VVFLHVTYV, SARS-CoV-derived spike (S) and nucleocapsid (N) protein T cell epitopes that are identical in SARS-CoV-2 including one or more of FIAGLIAIV, GLIAIVMVTI, IITTDNTFV, ALNTLVKQL, LITGRLQSL, LLLQYGSFC, LQYGSFCT, NLNESLIDL, RLDKVEAEV, RLNEVAKNL, RLQSLQTYV, VLNDILSRL, VVFLHVTYV, ILLNKHID, FPRGQGVPI, LLLLDRLNQ, GMSRIGMEV, ILLNKHIDA, ALNTPKDHI, LALLLLDRL, LLLDRLNQL, LLLLDRLNQL, LQLPQGTTL, AQFAPSASA, TTLPKGFYA, VLQLPQGTTL, GYQPYRVVVL, PYRVVVLSF, LSPRWYFYY, DSFKEELDKY, LIDLQELGKY, PYRVVVLSF, GTTLPKGFY, VTPSGTWLTY, GSFCTQLNR, GVVFLHVTY, AQALNTLVK, MTSCCSCLK, ASANLAATK, SLIDLQELGK, SVLNDILSR, TQNVLYENQK, CMTSCCSCLK, VQIDRLITGR, KTFPPTEPK, KTFPPTEPKK, LSPRWYFYY, ASAFFGMSR, ATEGALNTPK, QLPQGTTLPK, QQQGQTVTK, QQQQGQTVTK, SASAFFGMSR, SQASSRSSSR, TPSGTWLTY, FPNITNLCPF, APHGVVFLHV, FPRGQGVPI, APSASAFFGM, GAALQIPFAMQMAYR, GWTFGAGAALQIPFA, IDRLITGRLQSLQTY, ISGINASVVNIQKEI, LDKYFKNHTSPDVDL, LGDISGINASVVNIQ, LGFIAGLIAIVMVTI, LNTLVKQLSSNFGAI, LQDVVNQNAQALNTL, LQSLQTYVTQQLIRA, LQTYVTQQLIRAAEI, AQKFNGLTVLPPLLT, PCSFGGVSVITPGTN, QIPFAMQMAYRFNGI, QQLIRAAEIRASANL, QTYVTQQLIRAAEIR, AYRFNGIGVTQNVLY, SSNFGAISSVLNDIL, TGRLQSLQTYVTQQL, WLGFIAGLIAIVMVT, CVNFNFNGLTGTGVL, DKYFKNHTSPDVDLG, IDAYKTFPPTEPKKD, MSRIGMEVTPSGTWL, NKHIDAYKTFPPTEP, VLQLPQGTTLPKGFY, LQIPFAMQM, RVDFCGKGY, YEQYIKWPWY, GRLQSLQTY, or VRFPNITNL, SARS-CoV-derived linear B cell epitopes that are identical in SARS-CoV-including DVVNQNAQALNTLVKQL, EAEVQIDRLITGRLQSL, EIDRLNEVAKNLNESLIDLQELGKYEQY, EVAKNLNESLIDLQELG, GAALQIPFAMQMAYRFN, GAGICASY, AISSVLNDILSRLDKVE, GSFCTQLN, ILSRLDKVEAEVQIDRL, KGIYQTSN, AMQMAYRF, KNHTSPDVDLGDISGIN, MAYRFNGIGVTQNVLYE, AATKMSECVLGQSKRVD, PFAMQMAYRFNGIGVTQ, QALNTLVKQLSSNFGAI, QLIRAAEIRASANLAAT, QQFGRD, RASANLAATKMSECVLG, RLITGRLQSLQTYVTQQ, EIDRLNEVAKNLNESLIDLQELGKYEQY, SLQTYVTQQLIRAAEIR, or DLGDISGINASVVNIQK, SARS-CoV-derived discontinuous B cell epitopes that have at least one site with an identical amino acid to the corresponding site in SARS-CoV-2 including epitopes with IEDB ID 910052, 77444, or 77442, or 7D10 epitope.

19. The method of claim 1, wherein the extraction solvent comprises at least one of alcohols, hydrocarbons, amines, ethers, fluoroethers, surfactants, detergents, or combinations thereof.

20. The method of claim 1, wherein the extraction solvent comprises sevoflurane.
